SEQUENCE LISTING (13 sequences)

SEQ ID NO: 1 - protein, 408 aa, Homo sapiens
MAPWPHENSSLAPWPDLPTLAPNTANTSGLPGVPWEAALAGALLALAVLATVGGNLLVIVAIAWTPRLQTMTNVFVTSLAAADLVMGLLVVPPAATLALTGHWPLGATGCELWTSVDVLCVTASIETLCALAVDRYLAVTNPLRYGALVTKRCARTAVVLVWVVSAAVSFAPIMSQWWRVGADAEAQRCHSNPRCCAFASNMPYVLLSSSVSFYLPLLVMLFVYARVFVVATRQLRLLRGELGRFPPEESPPAPSRSLAPAPVGTCAPPEGVPACGRRPARLLPLREHRALCTLGLIMGTFTLCWLPFFLANVLRALGGPSLVPGPAFLALNWLGYANSAFNPLIYCRSPDFRSAFRRLLCRCGRRLPPEPCAAARPALFPSGVPAARSSPAQPRLCQRLDGASWGVS

SEQ ID NO: 2 - protein, 89 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 3 - protein, 360 aa, Homo sapiens [sequence omitted]

SEQ ID NO: 4 - DNA, 20 bp, Artificial
  Forward primer for ADRB2
ctggttgggctacgtcaact

SEQ ID NO: 5 - DNA, 20 bp, Artificial
  Reverse primer for ADRB2
cttccttgggagtcaacgct

SEQ ID NO: 6 - DNA, 20 bp, Artificial
  Forward primer for ADRB3
gcaggaggaagatggaaacc

SEQ ID NO: 7 - DNA, 20 bp, Artificial
  Reverse primer for ADRB3
cagttactggagacacccgc

SEQ ID NO: 8 - DNA, 20 bp, Artificial
  Forward primer for CNR1
cgttgagcctggcctaatca

SEQ ID NO: 9 - DNA, 20 bp, Artificial
  Reverse primer for CNR1
gaaccaacggggagttgtct

SEQ ID NO: 10 - DNA, 20 bp, Artificial
  Forward primer for CNR2
ttcgcccacgcttagtgatt

SEQ ID NO: 11 - DNA, 20 bp, Artificial
  Reverse primer for CNR2
agctggtgcaggaattcaca

SEQ ID NO: 12 - DNA, 20 bp, Artificial
  Forward primer for ACTB
ctgtcgagtcgcgtccaccc

SEQ ID NO: 13 - DNA, 20 bp, Artificial
  Reverse primer for ACTB
gctttgcacatgccggagcc

(19) United States
(12) Patent Application Publication
(10) Pub. No.: US 2017/0355959 A1
(43) Pub. Date: Dec. 14, 2017

(54) METHODS FOR MOBILISING POPULATIONS OF STEM CELLS

(71) Applicant: Imperial Innovations Limited, London (GB)

(72) Inventors: Sara RANKIN, London (GB); Tariq GHAZOUANI FELLOUS, Pozzuoli NA Campania (IT)

(21) Appl. No.: 15/537,672

(22) PCT Filed: Dec. 21, 2015
(86) PCT No.: PCT/GB2015/000330

§ 371 Date: Jun. 19, 2017

(30) Foreign Application Priority Data
Dec. 19, 2014 (GB) 1422828.2

(51) Int. Cl. C12N 5/0789; A61K 31/4178; C12N 5/0775; A61K 35/28; G01N 33/50; A61K 45/06

(52) U.S. Cl. CPC C12N 5/0647; G01N 33/5073; A61K 45/06; C12N 5/0663; A61K 35/28; A61K 31/4178

(57) ABSTRACT

The invention provides a method for mobilising haematopoietic stem and progenitor cells (HSPC) and/or mesenchymal stem cells (MSC) in a subject, the method comprising administering a selective beta-3 adrenergic receptor (AR) agonist and an inhibitor of the CXCR4/CXCL12 chemokine axis to the subject.

The invention relates to methods for mobilising populations of stem cells and in particular haematopoietic stem and progenitor cells and mesenchymal stem cells.

The listing or discussion of a prior-published document in this specification should not necessarily be taken as an acknowledgement that the document is part of the state of the art or is common general knowledge.

Haematopoietic stem and progenitor cells (HSPCs) are bone marrow derived stem and progenitor cells that form the cellular constituents of blood (eg erythrocytes and leucocytes). They include both haematopoietic stem cells (HSCs) and haematopoietic progenitor cells (HPCs), and have the capacity to reconstitute bone marrow that has previously been ablated by radiation. HSPCs are used for bone marrow transplants (BMTs). To obtain populations of HSPCs, the cells are harvested from donors by mobilising the cells into the blood. Currently, the cytokine G-CSF is used to mobilise HSPCs (Cashen et al, 2004, Curr Haem Rep 3: 406-412) and it has been shown that maximal mobilisation requires treatment daily over 3-5 days, with blood being collected on day 5. However, in approximately 20% of patients not enough HSPCs are mobilised to perform a BMT. Thus, there is a need to find more effective reagents for mobilising HSPCs.

HSPCs express the receptor CXCR4 and migrate in response to the chemokine stromal cell-derived factor (SDF)-1-alpha (CXCL12) (Aiuti et al, 1997, J Exp Med 185: 111-120). CXCR4 is the receptor for CXCL12, which is expressed constitutively in the bone marrow. It is well accepted that the CXCR4/CXCL12 chemokine axis acts as a critical retention mechanism for HSPCs in the bone marrow (Levesque and Winkler, 2008; Vagima et al, 2011). G-CSF has been shown to disrupt this axis by reducing levels of CXCL12 in the bone marrow. A role for both myeloid cells and the sympathetic nervous system (SNS) has been implicated in this response (Chow et al, 2011; Christopher et al, 2011; Katayama et al, 2006). Likewise, CXCR4 antagonists have been used to directly disrupt this chemokine axis and mobilize HSPCs (Broxmeyer et al, 2005; Fricker, 2013; Martin et al, 2006). For example, AMD3100, a specific CXCR4 antagonist, stimulates a rapid rise in circulating numbers of HSPCs in both mice and humans (Hatse et al, (2002) FEBS Letters 527: 255-262; Liles et al, (2003) Blood 102: 2728-2730; Broxmeyer et al, (2005) J. Exp. Med. 201: 1307-1318). Of note, maximal mobilisation of HSPCs following administration of the CXCR4 antagonists occurs within 1-2 hours, while maximal mobilisation with G-CSF takes 3-5 days with daily administration of G-CSF. Further, we and others have reported synergistic mobilisation of HSPCs into the blood when the CXCR4 antagonist is administered following a 4 day G-CSF pre-treatment, which has provided a strategy to mobilise HSPCs in patients that are identified in the clinic as ‘poor mobilisers’ (Martin et al, (2006) Br. J. Haem. 134: 326-329; Broxmeyer et al, (2005) J. Exp. Med. 201: 1307-1318; Flomenberg et al, (2005) Blood 106: 1867-1874).

In addition to the pharmacological (enforced) mobilisation of HSPCs, it is well known that these stem cells are mobilised in response to circadian oscillations (Mendez-Ferrer et al, 2008). This response is governed by sympathetic nerves that innervate the bone marrow. Activation of beta-2 adrenoreceptors is linked to the up-regulation of clock genes, while stimulation of beta-3 adrenoreceptors on osteoblasts is associated with a rapid reduction in their production of CXCL12 (Mendez-Ferrer et al, 2010).

Mesenchymal stem cells (MSCs) are another type of bone marrow-derived stem cell that have the capacity to differentiate into the major cellular components of the skeleton, namely osteocytes, chondrocytes and adipocytes (Bianco et al, 2013). Due to their release of paracrine factors, they have been shown to reduce inflammation and enhance tissue regeneration in a wide range of animal models of disease (eg graft versus host disease (GvHD), ischaemic heart disease (IHD) and acute respiratory distress syndrome (ARDS)) and are now in clinical trials for various diseases. A review of the current and potential clinical uses of MSCs is provided in Giordano et al (2007) J. Cell. Physiol. 211: 27-35. In most clinical studies, MSCs are administered intravenously and are presumed to traffic to sites of tissue injury or damage, but few studies have addressed whether endogenous MSCs can circulate and traffic to sites of tissue damage or disease. An increase in circulating MSPCs has been reported in animal models of injury, and high-mobility group protein B1 (HMGB1) and substance P have been identified as the factors that mediate this response (Hong et al, 2009; Lan et al, 2012; Rochefort et al, 2006; Tamai et al, 2011). Furthermore, an increase in circulating stromal cells has been reported in blood from patients following myocardial infarct (Iso et al, 2012; Wang et al, 2006) or burns (Hu et al, 2013).

Human MSCs are typically defined as cells that are:
1. Plastic adherent (ie adherent to typical plastic cell culture vessels);
2. Triple positive for the cell surface markers CD105, CD73 and CD90;
3. Negative for the cell surface markers, CD34, CD45, CD14, CD11b and CD19; and
4. Exhibit tri-lineage differentiation.

Murine MSCs exhibit 1 and 4 (above), but the CD markers may be different (for references see above).

MSCs represent <0.001% of bone marrow cells (Tondreau et al, (2005) Stem Cells 23: 1105-1112). Isolation requires ex vivo expansion of MSCs in fetal calf serum (FCS) containing growth medium. There are a number of problems associated with such isolation and expansion procedures, including the use of FCS, the potential of MSCs to transform and the possibility that such ex vivo culture may reduce the functionality of these cells. For example, see Procock D & Olson (2007) Blood. 109(8): 3147-51 (Epub 2006 Dec. 14), where page 3150 indicates that MSCs expanded ex vivo may form tumours in vivo.

The factors regulating the mobilization of MSCs are currently unknown. We have previously examined whether pharmacological strategies known to mobilise HSPCs from the bone marrow into the blood can also mobilise MSCs; however, G-CSF and/or the CXCR4 antagonist were not effective in this respect (Pitchford et al, 2009). In contrast, we have reported that MSCs, but not HSPCs, can be mobilised into the blood when a CXCR4 antagonist is administered to mice pre-treated with VEGF (Pitchford et al, 2009). This suggests that distinct mechanisms may regulate the mobilisation of these discrete populations of stem cells.

Given the diverse uses of bone marrow derived stem cells such as HSPCs and MSCs, there is a need to develop effective strategies for mobilising them. In the present application, we identify factors that are effective in mobilising HSPCs and/or MSCs, and which are therefore useful in the clinical applications described above.

A first aspect of the invention provides a method for mobilising haematopoietic stem and progenitor cells (HSPC) and/or mesenchymal stem cells (MSC) in a subject, the method comprising administering a selective beta-3 adrenergic receptor (AR) agonist and an inhibitor of the CXCR4/CXCL12 chemokine axis to the subject.

This aspect of the invention also includes a selective beta-3 AR agonist and an inhibitor of the CXCR4/CXCL12 chemokine axis, for use in mobilising HSPCs and/or MSCs in a subject. Similarly, this aspect of the invention also includes the use of a selective beta-3 AR agonist and an inhibitor of the CXCR4/CXCL12 chemokine axis, in the manufacture of a medicament for mobilising HSPCs and/or MSCs in a subject.

The subject may be any individual, for example a human or mammalian individual, such as a horse, dog, pig, cow, sheep, rat, mouse, guinea pig or primate. Preferably, the subject is a human individual.

By mobilising HPSCs and/or MSCs, we include the meaning of the HSPCs and/or MSCs being mobilised from the bone marrow of a subject, for example into the blood (eg peripheral blood). In this way, the number of HPSCs and/or MSCs in the blood will increase, thus allowing for a more accessible source of stem cells for use in transplantation. However, it will be appreciated that MSCs may also be mobilised from mesenchymal tissue, adipose tissue, liver, tendons, synovial membrane, amniotic fluid, placenta, umbilical cord and teeth.

Mobilisation of stem cells from the bone marrow may be assessed by routine methods in the art, including those described in the Examples below. Conveniently, peripheral blood is harvested from a subject, red blood cells are lysed, and the remaining cells are subject to colony forming unit (CFU) assays to enumerate HSPCs or MSCs. For example, for HSPCs, cells may be added to Methocult medium (M3434, purchased from Stem cell technologies), incubated at 37° C. and quantified after 11-12 days; for MSCs, cells may be added to tissue culture treated plates containing Mesencult proliferation kit medium (Stem cell technologies), incubated at 37° C. and quantified after 20-21 days. It will be appreciated that the CFUs may be further expanded and assessed for lineage markers by flow cytometry, mesenchymal trilineage differentiation and in vivo bone formation.

Typically, the method results in mobilisation of HSPCs and/or MSCs from the bone marrow such that the level of HSPCs and/or MSCs in the peripheral blood increases by at least 2-fold, such as at least 4-fold, 5-fold, 6-fold, 7-fold or 8-fold. However, any level of increase is encompassed by the scope of the invention. Generally, the level of HSPCs and/or MSCs is increased in the peripheral blood within 1-2 hours.

By a selective beta-3 AR agonist, we include the meaning of any molecule that mimics or upregulates (eg increases, potentiates or supplements) an activity of the beta-3 adrenergic receptor, to a greater extent than it mimics or upregulates an activity of any other adrenergic receptor, including any other beta adrenergic receptor (eg beta-1 AR or beta-2 AR) or any alpha adrenergic receptor, or any other protein. Generally, the beta-3 AR agonist is a molecule that mimics or upregulates an activity of the beta-3 adrenergic receptor at least 1.5, 2.0, 2.5, 5.0, 10, 15 and 20 times more than it does an activity of another adrenergic receptor or any other protein. Preferably, the beta-3 AR agonist does not have an effect on an activity of any other protein, such as any other beta-adrenergic receptor (eg beta-1 AR or beta-2 AR) or any alpha adrenergic receptor.

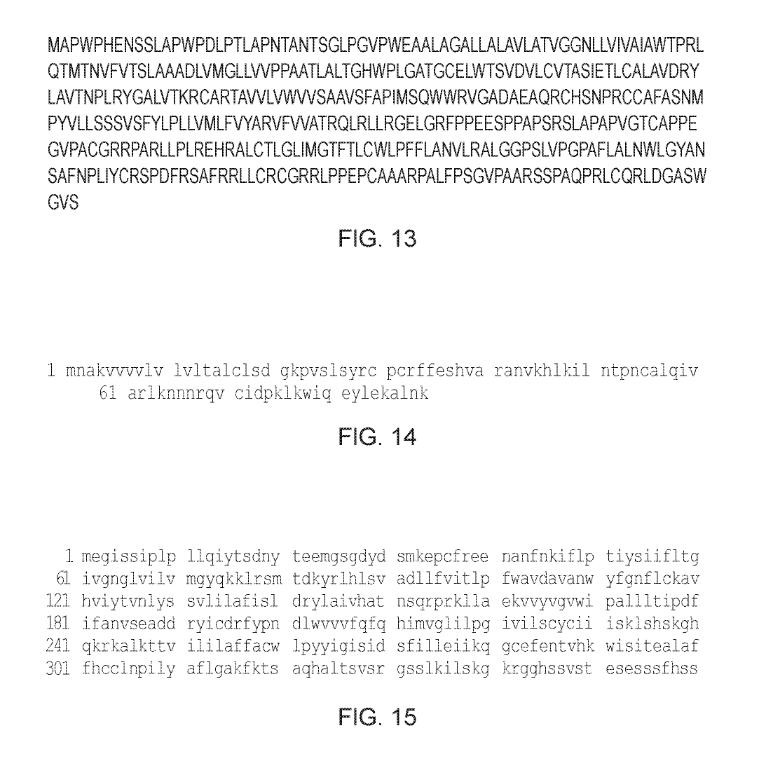

The amino acid sequence of human beta-3 AR is provided in FIG. 13 (SEQ ID No: 1) and has GenBank accession no: NP_000016. It will be appreciated that this sequence serves only to identify the relevant protein and that any natural variation and polymorphisms of this sequence is encompassed by the invention. The invention also includes beta-3 AR proteins in other species that have orthologous sequences to that in FIG. 13, for example beta-3 AR proteins from horse, dog, pig, cow, sheep, rat, mouse, guinea pig or a primate. It will be appreciated, that when the method is used to mobilise HSPCs and/or MSCs from a particular subject, the method preferably comprises administering an agonist of the beta-3 AR of that subject. Thus, when the subject is human, an agonist of human beta-3 AR is used, and so on.

It is envisaged that selective agonism of beta-3 AR may reduce unwanted side effects in a subject by preventing agonism of unnecessary targets. The Examples below indicate that the action of the general beta AR agonist, isoproterenol, is via beta-3 AR not beta-2 AR. Thus, the reduction of beta-2 AR and/or beta-1 AR agonism by using a selective beta-3 AR agonist may increase efficacy and reduce unwanted side effects.

The agonist may be an endogenous agonist such as natural ligand of the beta-3 AR, or it may be an exogenous agonist, such as a synthetic ligand or molecule (eg drug) or a recombinant protein. It will be appreciated that the agonist does not necessarily need to be one that binds to the beta-3 AR, but can merely serve to replicate the activity of signalling through the beta-3 AR. Typically, however, the beta-3 AR agonist is one that binds to the beta-3 AR, in which case it typically binds to the beta-3 AR to a greater extent than it binds to any other adrenergic receptor, including any other beta adrenergic receptor (eg beta-1 AR or beta-2 AR) or any alpha adrenergic receptor, or any other protein. Preferably, the beta-3 AR agonist does not bind to any other adrenergic receptor, including any other beta adrenergic receptor (eg beta-1 AR or beta-2 AR) or any alpha adrenergic receptor, or any other protein.

The agonist may be a small molecule (synthetic or naturally occurring), a peptide, a peptidomimetic, a protein, an antibody, a nucleic acid (eg DNA or RNA) or a carbohydrate.

By a selective beta-3 AR agonist, we also include the meaning of two or more molecules having the property of selective beta-3 AR agonism.

Various selective beta-3 AR agonists have been identified and are known in the art. Examples include BRL37344, Mirabegron and CL316243. Other examples include Amibegron (SR-58611A) (Consoli et al Pharmacology 573 (1-3): 139-47; Overstreet et al Pharmacology, Biochemistry, and Behavior 89 (4): 623-6); CL-316,243 (Fu et al European Journal of Pharmacology 584 (1): 202-6); L-742,791 (Candelore et al, The Journal of Pharmacology and Experimental Therapeutics 290 (2): 649-55); L-796,568 (Larsen et al Clinical Nutrition 76 (4): 780-8); LY-368,842; Mirabegron (YM-178) (Gras et al Drugs of today (Barcelona, Spain: 1998) 48 (1): 25-32); Ro40-2148; Solabegron (GW-427,353) (Hicks et al (The Journal of Pharmacology and Experimental Therapeutics 323 (1): 202-9); Betanis (Astellas); Betmiga (Astellas); Myrbetriq (Astellas); TT-138 (Mitsubishi Tanabe Pharma); GS-332 (Mitsubishi Tanabe Pharma); MN-246 (Mitsubishi Tanabe Pharma); FMP-1970302 (Molecular Design); and 4SC (4sc discovery).

It will be appreciated that the skilled person can readily identify other beta-3 AR agonists without undue burden, for example by assessing the effect of candidate molecules on an activity of the beta-3 AR agonist receptor, and/or assessing the binding of candidate molecules to the beta-3 AR receptor. Beta-3 adrenergic receptors are primarily expressed on adipocytes and are known to regulate lipolysis and thermogenesis. Thus, in adipocytes, beta-3 AR agonists stimulate lipase induced hydrolysis of triglycerides, leading to the generation of glycerol and free fatty acids that are detectable in plasma after 3-4 days of treatment. Beta-3 adrenergic receptors have also been reported on stromal cells, where their activation has been shown to reduce CXCL12 production by these cells. Thus, it would be a matter of routine for the skilled person to find other suitable beta-3 AR agonists, for instance by assessing the effect of candidate molecules on the activity of adipoctyes in regulating lipolysis or thermogenesis.

For the avoidance of doubt, by a selective beta-3 AR agonist, we also include the meaning of prodrugs thereof. For example, the beta-3 AR agonist may be administered as a prodrug which is metabolised or otherwise converted into its active form once inside the body of a subject. Thus, the method of the invention includes administration of a prodrug of a selective beta-3 AR agonist. The term “prodrug” as used in this application refers to a precursor or derivative form of a pharmaceutically active substance that is less active than the parent drug and is capable of being enzymatically activated or converted into the more active parent form. This may be useful to improve the selectivity of the drug so that it acts predominantly on its intended target (eg cell or tissue).

By an inhibitor of the CXCR4/CXCL12 chemokine axis we include the meaning of any agent that inhibits (eg decreases or abolishes) signalling through the CXCR4 receptor.

In one embodiment, the agent may act to decrease the expression or function of the chemokine, CXCL12, also known as SDF-1 (eg the binding of the chemokine to its receptor CXCR4 and further signalling). By decrease expression, we include the meaning of an agent that downregulates expression of the gene encoding CXCL12 or which reduces the amount of expressed CXCL12 protein.

Alternatively, and in a preferred embodiment, the agent may act to block or antagonise the chemokine receptor, CXCR4.

By an inhibitor of the CXCR4/CXCL12 chemokine axis, we also include the meaning of two or more molecules having the property of inhibiting the CXCR4/CXCL12 chemokine axis. Thus, two or more inhibitors may be used such as two or more agents that act to decrease the expression or function of the chemokine, CXCL12, or two or more agents that act to block or antagonise the chemokine receptor, CXCR4, or at least one agent that acts to decrease the expression or function of the chemokine, CXCL12 and at least one agent that acts to block or antagonise the chemokine receptor CXCR4.

The amino acid sequence of human CXCL12 is provided in FIG. 14 (SEQ ID No: 2) and has GenBank accession number CAG29279. The alpha isoform has GenBank accession no NP_954637, the beta isoform has GenBank accession no NP_000600, and the gamma isoform has GenBank accession number NP_001029058 (see also UniProt P48061). The human amino acid sequence of CXCR4 is provided in FIG. 15 (SEQ ID No: 3) and has GenBank accession number CAA12166.1 (see also UniProt P61073). Again, these sequences serve only to identify the referenced proteins and it will be appreciated that any natural variation and polymorphisms of these sequences is encompassed by the invention. Also encompassed are orthologues of the listed sequences. Preferably, when the method is used to mobilise HSPCs and/or MSCs from a particular subject, the inhibitor of the CXCR4/CXCL12 chemokine axis is an inhibitor of the CXCR4/CXCL12 axis in that subject. For example, when mobilising HSPCs and/or MSCs in a human, an inhibitor of the CXCR4/CXCL12 axis in humans is used, and so on.

The agents that decrease the expression or function of CXCL12 or that block or antagonise CXCR4 may be selected from the group consisting of small molecules (synthetic or naturally occurring), peptides, peptidomimetics, polypeptides, antibodies, nucleic acids (eg DNA or RNA), and carbohydrates.

In more particular embodiments, the polypeptides that decrease the expression of CXCL12 may be selected from the group consisting of a cytokine, a colony stimulating factor, a protease or a chemokine other than CXCL12. The protease may be a dipeptidyl peptidase-1 (DDP-1 OR CD26). The chemokine other than CXCL12 may be selected from the group consisting of IL-8, MIP-1[alpha] and Gro[beta]. The colony stimulating factor may be selected from the group consisting of granulocyte colony stimulating factor (G-CSF), granulocyte-macrophage colony stimulating factor (GM-CSF), macrophage colony stimulating factor (M-CSF), stem cell factor, FLT-3 ligand or a combination thereof. The nucleic acid may be a DNA or an RNA molecule. The nucleic acid may be a small interfering RNA (siRNA) molecule or an antisense molecule specific for CXCL12 or CXCR4. The carbohydrate may be a sulfated carbohydrate selected from the group consisting of Fucoidan and sulfated dextran.

Preferably, the inhibitor of the CXCR4/CXCL12 chemokine axis acts to block or antagonise the chemokine receptor, CXCR4. Thus, the inhibitor may downregulate (eg suppress or inhibit) at least one activity of CXCR4. The antagonist may be an agent that inhibits or decreases the interaction between CXCR4 and its ligand CXCL12, or it may be an agent that inhibits or decreases the interaction between and CXCR4 and some other molecule required for its activity. By antagonist, we also include the meaning of an agent that down-regulates expression of the gene encoding CXCR4 or which reduces the amount of expressed CXCR4 protein, such as an siRNA or antisense molecule specific for CXCR4.

In a preferred embodiment, the inhibitor of the CXCR4/CXCL12 chemokine axis is the CXCR4 antagonist AMD-3100 or its analogues, derivatives or combinations thereof. Alternative names for AMD-3100 include plerixafor and mozobil. The structure of AMD-3100 and its derivatives and analogues thereof may be found in U.S. Pat. No. 6,987,102, which is incorporated by reference in its entirety. In another embodiment, the CXCR4 antagonist is KRH3955 (Murakami et al, 2009, Antimicrob Agents Chemother 53(7): 2940-8). Other CXCR4 antagonists are also in development. Examples of CXCR4 antagonists are provided in WO 01/85196; WO 00/09152; WO 99/47158; Ichiyama et al (2003) PNAS USA 100: 4185-4190; WO 01/16161; WO 01/56591; WO 01/85196; WO 2004/024178; WO 2006/074426; WO 2005/121123; WO 2006/074428; WO 2004/096840; WO 2007/022385; WO 02/094261; WO 2007/064620; WO 2006/126188; and WO 99/50461, which are incorporated herein by reference. Yet further examples of CXCR4 antagonists, either launched or in development, include (i) plerixafor; AMD-2763; GZ-316455; JM3100; Mozobil; SDZ-SID-791; or SID791 (originator. Sanofi); (ii) RPI-MN, alpha-cobratoxin, HIV, ReceptoPha (IM); alpha-cobratoxin, HIV, ReceptoPha (oral); alpha-cobratoxin, HIV, ReceptoPharm; alpha-cobratoxin, neurodegenrativ, ReceptoPharm; ALS therapy, ReceptoPharm; ALS therapy,. ReceptoPharm (IM); ALS therapy, ReceptoPharm (oral); HIV therapy, ReceptoPharm; HIV therapy, ReceptoPharm (IM); HIV therapy, ReceptoPharm (oral); neurodegenerative therapy, ReceptoPharm; Pepteron; Pepteron (IM); Pepteron (oral); rabies therapy, ReceptoPharm; rabies therapy, ReceptoPharm (IM); rabies therapy, ReceptoPharm (oral); rheumatoid arthritis ther, Rec (IM); rheumatoid arthritis ther, Rec (oral); rheumatoid arthritis therapy, ReceptoPharm; RPI-MN (IM); or RPI-MN (oral) (originator: Nutra Pharma); (iii) POL6326 (originator: Polyphor); (iv) OHR-118; AVR-118; AVR-118 (injectable); AVR-118 (oral); Panar 097; Product R; Product R; ADVR; Reticulose; reticulose, ADVR; or Substance R (originator: Advanced Viral Research); (v) LY-2510924 (Eli Lilly); (vi) burixafor; CXCR4 antagonist, TaiGen; or TG0054 (originator: TaiGen Biotechnology); (vii) BKT-140, BL-8040, or T140 (originator: Biokine Therapeutics); (viii) ulocuplumab, BMS936564, or MDX1338 (originator: Bristol-Myers Squibb); (ix) filgrastim+plerixafor; or UMK121 (originator. Proteonomix); (x) GMI-1257; E-selectin/CXCR4 antagonist, GlycoMimetics; or GMI-1215 (GlycoMimetics); (xi) CXCR4-ZFN, T-cell; or ZFN-CXCR4, T-cell (originator. Sangamo BioSciences); (xii) CXCR4 compounds, Ligand; or CXCR4 compounds, Proximagen (originator: Ligand); (xiii) CXCR-4 antagonist (originator: Heptares); and (xiv) CF172 (originator: Roche).

It will be appreciated that the skilled person can readily identify other inhibitors of the CXCL12/CXCR4 chemokine axis without undue burden, for example by assessing the effect of candidate molecules on either the expression or function of CXCL12 (eg ability to bind to the CXCR4 receptor) or on signalling through the CXCR4 receptor. Screening assays to determine the effect of candidate molecules on protein expression or signalling function are very well known in the art and typically involve the use of standard reporter assays.

In a particular embodiment when the methods of the invention are used to mobilise HSPCs, administering an inhibitor of the CXCR4/CXCL12 chemokine axis may comprise administering G-CSF and a CXCR4 antagonist. Hence, the method of mobilising HSPCs may comprise administering a beta AR agonist (eg a selective beta-3 AR agonist), G-CSF and a CXCR4 antagonist.

The invention includes a method for mobilising HSPCs and/or MSCs in a subject, the method comprising administering an inhibitor of the CXCR4/CXCL12 chemokine axis to the subject, wherein the subject is one who is administered a selective beta-3 AR agonist.

The invention also includes a method for mobilising HSPCs and/or MSCs in a subject, the method comprising administering a selective beta-3 AR agonist to the subject, wherein the subject is one who is administered an inhibitor of the CXCR4/CXCL12 chemokine axis.

A second aspect of the invention provides a method for mobilising mesenchymal stem cells (MSC) in a subject, the method comprising administering a beta adrenergic receptor (AR) agonist (eg a selective beta-3 AR agonist), and an inhibitor of the CXCR4/CXCL12 chemokine axis to the subject.

This aspect of the invention also includes a method for mobilising mesenchymal stem cells (MSC) in a subject, the method comprising administering a beta AR agonist to the subject, wherein the subject is one who is administered an inhibitor of the CXCR4/CXCL12 chemokine axis. This aspect of the invention also includes a method for mobilising mesenchymal stem cells (MSC) in a subject, the method comprising administering an inhibitor of the CXCR4/CXCL12 chemokine axis to the subject, wherein the subject is one who is administered a beta AR agonist.

Preferences for the mobilisation of MSCs, the subject, and the inhibitor of the CXCR4/CXCL12 chemokine axis include those described above in relation to the first aspect of the invention.

By beta AR agonist, we include the meaning of meaning of any molecule that mimics or upregulates (eg increases, potentiates or supplements) an activity of the beta adrenergic receptor, to a greater extent than it mimics or upregulates an activity of any other adrenergic receptor, including any alpha adrenergic receptor, or any other protein.

Generally, the beta AR agonist is a molecule that mimics or upregulates an activity of a beta adrenergic receptor at least 1.5, 2.0, 2.5, 5.0, 10, 15 and 20 times more than it does an activity of another adrenergic receptor or any other protein. Preferably, the beta AR agonist does not have an effect on an activity of any other protein, such as any other adrenergic receptor including any alpha adrenergic receptor.

The agonist may be a small molecule (synthetic or naturally occurring), a peptide, a peptidomimetic, a protein, an antibody, a nucleic acid (eg DNA or RNA) or a carbohydrate.

In one embodiment, the beta AR agonist is a general beta AR agonist. In other words, the beta AR agonist does not exhibit any selectivity in binding or agonism between the different isoforms of the beta AR, such as between the beta-1, beta-2 and beta-3 isoforms. Various general beta AR agonists have been identified and are known in the art. Examples include isoproterenol, epinephrine, and norepinephrine.

In a preferred embodiment, however, the beta AR agonist is a selective beta-3 AR agonist including any of those defined and described above. Preferably, the selective beta-3 AR agonist is BRL37344, Mirabegron or CL316243. Other examples include Amibegron (SR-58611A); CL-316,243; L-742,791; L-796,568; LY-368,842; Mirabegron (YM-178); Ro40-2148; Solabegron (GW-427,353); Betanis (Astellas); Betmiga (Astellas); Myrbetriq (Astellas); TT-138 (Mitsubishi Tanabe Pharma); GS-332 (Mitsubishi Tanabe Pharma); MN-246 (Mitsubishi Tanabe Pharma); FMP-1970302 (Molecular Design); and 4SC (4sc discovery).

A third aspect of the invention provides a method for mobilising haematopoietic stem and progenitor cells (HSPC) and/or mesenchymal stem cells (MSC) in a subject, the method comprising administering a beta adrenergic receptor (AR) agonist and an inhibitor of the CXCR4/CXCL12 chemokine axis to the subject, wherein a beta AR agonist is administered before the inhibitor of the CXCR4/CXCL12 chemokine axis.

Preferences for the mobilisation of HSPCs and/or MSCs, the subject, the inhibitor of the CXCR4/CXCL12 chemokine axis include those described above in relation to the first aspect of the invention, and preferences for the beta AR agonist include those described above in relation to the second aspect of the invention. Preferably, the beta AR agonist is a selective beta-3 AR agonist including any of those described in relation to the first aspect of the invention.

By “administered before the inhibitor of the CXCR4/CXCL12 chemokine axis”, we include the meaning that before the inhibitor of the CXCR4/CXCL12 chemokine axis is administered, the subject is administered a beta AR agonist. For example, the beta AR agonist may be administered at least 1 hour before the inhibitor of the CXCR4/CXCL12 chemokine axis, such as at least 2 hours, 5 hours, 10 hours or 12 hours before, or the beta AR agonist may be administered at least 1 day before, such as at least 2 days, 3 days, 4 days or 5 days before the inhibitor of the CXCR4/CXCL12 chemokine axis. As is discussed further below, the beta AR agonist may be administered chronically to the subject, wherein it is administered to the subject at regular intervals or continuously over a given period. In this case, it will be appreciated that the commencement of the beta AR agonist administration would be before (eg at least 2 hours before or at least 4 days before) the inhibitor of the CXCR/CXCL12 chemokine axis.

In a particular embodiment, the beta AR agonist is administered (eg chronic administration is commenced to the subject) between 1 and 5 days before the inhibitor of the CXCR4/CXCL12 chemokine axis.

A fourth aspect of the invention provides a method for priming HSPCs and/or MSCs in a subject for mobilisation, the method comprising administering a beta AR agonist to the subject, wherein when the method is for priming HSPCs the beta AR agonist is a selective beta-3 AR agonist.

By priming HSPCs and/or MSCs in a subject for mobilisation, we include the meaning that the HSPCs and/or MSCs become responsive to an inhibitor of the CXCL12/CXCR4 chemokine axis (eg CXCR4 antagonist) such that in the presence of an inhibitor of the CXCL12/CXCR4 chemokine axis, they are mobilised from the bone marrow into the blood (eg peripheral blood).

Preferences for this and all subsequent aspects of the invention, for the subject, the inhibitor of the CXCR4/CXCL12 chemokine axis include those described above in relation to the first aspect of the invention, and preferences for the beta AR agonist include those described above in relation to the second aspect of the invention. Preferably, the beta AR agonist is a selective beta-3 AR agonist including any of those described in relation to the first aspect of the invention.

In an embodiment of any aspect of the invention, the beta AR agonist (eg the beta-3 AR agonist) is administered chronically to the subject. By ‘chronic administration’, we include the meaning of administration to the subject at regular intervals over a long period of time, for example over at least 1 hour, 2 hours, 0.5 days, 1 day, 2 days, 3 days, 4 days and 5 days, and preferably over at least 3-5 days such as over at least 4 days. This may include daily administration of the beta AR agonist (eg beta-3 AR agonist) over a period of several days, such as at least 2 days, 3 days, 4 days or 5 days. The frequency of dosage to the subject and the length of time over which the beta AR agonist is administered to the subject may be decided by the clinician with the benefit of clinical trials. This may depend on the physical condition of the subject. Other factors that may affect the dosage of beta AR agonist (eg beta-3 AR agonist) include the age (younger patients are known to be able to mobilise HSPCs better than older patients (Thomas et al (2002) Curr Opin Hematol 9: 183-9), gender, weight, build and fitness of the subject. The appropriate means of administration will depend on the beta AR agonist (eg beta-3 AR agonist) used, but could be administered intravenously, intraperitoneally, orally or subcutaneously. It will be appreciated that by chronic administration, we also include the meaning of continuous administration over a period of time, for example by way of a pump (eg minipump) or other appropriate means of continuous infusion.

In a further embodiment of any aspect of the invention, the inhibitor of the CXCL12/CXCR4 chemokine axis (eg CXCR4 antagonist) is administered acutely to the subject. By ‘acute administration’ we include the meaning of the administering the inhibitor of the CXCL12/CXCR4 chemokine axis (eg CXCR4 antagonist) over a short period of time, for example, over one or several hours or less than an one hour, but no more than 24 hours. It is preferred that the administration of the inhibitor of the CXCL12/CXCR4 chemokine axis (eg CXCR4 antagonist) is performed by way of a single administration such as a single injection. However, it may alternatively be administered through a number of administrations or may alternatively be a constant administration over said period of time. In one embodiment, the inhibitor of the CXCL12/CXCR4 chemokine axis (eg CXCR4 antagonist) is administered subcutaneously. An example of subcutaneous administration of the the inhibitor of the CXCL12/CXCR4 chemokine axis (eg CXCR4 antagonist) is provided in Hendrix et al (2000) Antimicrobial Agents and Chemotherapy 44: 1667-1673. Alternatively, the CXCR4 antagonist may be administered intraperitoneally as in Example 1, intravenously, orally or by any other appropriate means. The appropriate means of administration will depend on the inhibitor of the CXCL12/CXCR4 chemokine axis (eg CXCR4 antagonist) used and the condition or species of the subject. The length of time and the means for delivery may be decided by a clinician with the benefit of clinical trials. As with the administration of the beta AR agonist, the condition of the subject may affect the means of administration of the inhibitor of the CXCL12/CXCR4 chemokine axis.

In a particularly preferred embodiment, the beta AR agonist (eg selective beta-3 AR agonist) is administered chronically to the subject and the inhibitor of the CXCR4/CXCL12 chemokine axis (eg CXCR4 antagonist) is administered acutely to the subject. Most preferably, the beta AR agonist (eg selective beta-3 AR agonist) is administered before the inhibitor of the CXCR4/CXCL12 chemokine axis, and so the beta AR agonist (eg selective beta-3 AR agonist) may be administered chronically to the subject before the inhibitor of the CXCR4/CXCL12 chemokine axis is administered acutely to the subject. The beta AR agonist (eg selective beta-3 AR agonist) may be administered at regular intervals or continuously over a period of at least 1 hour, 2 hours, 0.5 days, 1 day, 2 days, 3 days, 4 days or 5 days before the inhibitor of the CXCR4/CXCL12 chemokine axis (eg CXCR4 antagonist) is administered acutely to the subject (eg over a period of 3-5 days).

It will be appreciated that any of the methods or uses described above in relation in the first, second, third and fourth aspects of the invention may be used to harvest HSPCs and/or MSCs.

The HSPCs and/or MSCs may be harvested from a body fluid using any suitable techniques known in the art. For example, the HSPCs and/or MSCs may be harvested from an extracted bone marrow or a perfused bone, for example using the methods described in Example 1, or other methods known in the art. This may have the advantage of yielding a more highly enriched population of HSPCs and/or MSCs than those harvested from circulating blood or other body fluid. Conveniently, however, HSPCs and/or MSCs are harvested from peripheral blood or another readily accessible body fluid. MSCs may be isolated simply by their adherence to plastic, or more selective techniques may be used. For example, a method for the isolation of human MSCs using a monoclonal antibody to Low affinity nerve growth factor receptor (LNGFR) is described in Jones et al (2006) Cytometry B Clin Cytom 70(B): 391-9. The methods of the invention may be carried out in the context of a clinical procedure of apheresis or leukopheresis, for example where white blood cells are separated from blood and blood is re-infused back into a subject.

Accordingly, a fifth aspect of the invention provides a method of obtaining a population of HSPCs and/or MSCs from a subject, the method comprising administering a beta adrenergic receptor (AR) agonist and an inhibitor of the CXCR4/CXCL12 chemokine axis to the subject so as to mobilise the HSPCs and/or MSCs from the bone marrow to the peripheral blood of the subject, and harvesting the mobilised HSPCs and/or MSCs from the peripheral blood; wherein when the method is for obtaining a population of HSPCs, the beta AR agonist is a selective beta-3 AR agonist.

In relation to harvesting MSCs, it will be appreciated that it is not essential to administer an inhibitor of the CXCR4/CXCL12 chemokine axis. This is because, as described further below, the inventors have found that administering a beta AR agonist (eg a selective beta-3 AR agonist) stimulates proliferation of MSCs, in the bone marrow. Hence, a sixth aspect of the invention provides a method of obtaining a population of MSCs from a subject, the method comprising administering a beta AR agonist (eg a selective beta-3 AR agonist), and harvesting MSCs from the bone marrow.

In relation to the fifth and sixth aspects of the invention, preferably, the beta AR agonist is a selective beta-3 AR agonist such as BRL37344, Mirabegron or CL316243, or any of those described above, for example in relation to the first or second aspects of the invention. It is also preferred that the inhibitor of the CXCL12/CXCR4 chemokine axis is an antagonist of CXCR4 such as AMD-3100.

In relation to the fifth and sixth aspects of the invention, preferably, the beta AR agonist is administered chronically and the inhibitor of the CXCL12/CXCR4 chemokine axis is administered acutely, and more preferably the beta AR agonist is administered at regular intervals or continuously over a period of at least 1 hour, 2 hours, 0.5 days, 1 day, 2 days, 3 days, 4 days or 5 days before the inhibitor of the CXCR4/CXCL12 chemokine axis is administered acutely to the subject.

The harvested HSPCs and/or MSCs may be cultivated, eg ex vivo or in vitro. This further step may facilitate the expansion of HSPCs and/or MSCs in populations of cells where they are in a minority. It may also be useful in samples from subjects that respond less well to the methods of the current invention. Additionally or alternatively, cultivation in vitro may allow modifications to be made to the cells as necessary depending on their intended use. For, example, the cultivated cells may be genetically modified. Genetic modification may lead to cells that are more efficacious or more compatible with the recipient of such cells (see, for example, Cucchiarini et al, Biomed Mater Eng 22(4): 197-208, 2012; and Shevchenko et al, J Transl Med 11: 138). The genetically modified cells may be more resistant to apoptosis than the wild-type cells. This could be achieved by the introduction of, or knockout of, genes involved in apoptotic mechanisms. It may also be achieved by fusion with another cell. For example, cultivated cells may be fused with immortal cells. Similarly, cultivated cells may also be fused with cells that express chemokine receptors or adhesion molecules, for example to facilitate trafficking to damaged tissues, as described further below.

Further applications of cultivating the cells harvested by the methods of the invention include (i) ex vivo stem cell expansion to supplement stem cell grafts with more immature precursors to shorten or potentially prevent hematopoietic cell depletion, including conditions such as pancytopenia, granulocytopenia, thrombocytopenia, anemia or a combination thereof; (ii) to increase the number of primitive progenitors to help ensure hematopoietic support for multiple cycles of high-dose therapy; (iii) to obtain sufficient number of stem cells from a single apheresis procedure, thus reducing the need for large-scale harvesting of marrow or multiple leukopheresis; (iv) to generate sufficient cells from a single cord-blood unit to allow reconstitution in an adult after high-dose chemotherapy; (v) to purge stem cell products of contaminating tumour cells; (vi) to generate large volumes of immunologically active cells with antitumour activity to be used in immunotherapeutic regimens, and (vii) to increase the pool of stem cells that could be targets for the delivery of gene therapy.

The cultivated cells may be administered back to the subject or to another subject. The use of cells in other subjects will be particularly beneficial in subjects who are unable to produce their own HSPCs and/or MSCs. It may be beneficial to use cells cultivated from the same subject for treatment of that subject to reduce the risk of rejection by the subject's immune response. The cultivation step may allow treatment of a subject with his own cells where that subject is unable to produce sufficient numbers of such cells due to a deficiency in their bone marrow. The deficiency in the subject's bone marrow may be as a result of chemotherapy, radiotherapy, an autoimmune disease, a genetic condition or any other such condition. Alternatively, the reintroduction of a subject's own cells after cultivation may be useful for targeted delivery of such cells to the intended site of action. For example, in the treatment of tissue damage following ischemia, cells could be targeted to the site of tissue damage. Re-introduction to the site of tissue damage/disease, may be appropriate for example in heart disease. The G-CSF clinical trials that have showed most promise are those where mobilised cells were isolated from the blood and then injected back into the heart by an intracoronary injection (Takano H (2007) Trends Pharm. Sci.,. 28(10): 512-7 (Epub 2007 Sep 20)).

It will be appreciated that HSPCs and/or MSCs mobilised and/or harvested by the methods of the current invention may be used for either autologous or allogenic stem cell transplants (i.e. either harvesting or mobilising cells from the patient to be put back into that same patient, or from donor to patient). Hence, in one example, the invention provides methods to enrich hematopoietic progenitor cells which are utilized in bone marrow (BM) and peripheral blood (PB) stem cell transplantation, wherein the hematopoietic stem cell transplantation (HSCT) protocols may for example be utilized for the purpose of treating the following diseases (from Ball, E. D., Lister, J., and Law, P. Hematopoietic Stem Cell Therapy, Chruchill Livingston (of Harcourt Inc.), New York (2000)): Aplastic Anemia; Acute Lymphoblastic Anemia.; Acute Myelogenous Leukemia; Myelodysplasia; Multiple Myeloma; Chronic Lymphocytic Leukemia; Congenital Immunodeficiencies (such as Autoimmune Lymphoproliferative disease, Wiscott-Aldrich Syndrome, X-linked Lymphoproliferative disease, Chronic Granulamatous disease, Kostmann Neutropenia, Leukocyte Adhesion Deficiency); Metabolic Diseases (for instance those which have been HSCT indicated such as Hurler Syndrome (MPS I/II), Sly NW Syndrome (MPS VII), Chilhood onset cerebral X-adrenoleukodystrophy, Globard_cell Leukodystrophy).

In one embodiment, the harvested cells obtained by the methods of the invention are stored in a cell bank. For example, the HSPCs and/or MSCs harvested by the methods of the invention may be stored in a bank for future use, typically in liquid nitrogen as is well known in the art. As an example, banked cells may be used if an individual has a heart attack in the future. There are many stem cell banks that have been set up to store cord blood stem cells (eg cells4life and futurehealth) and some that already offer to store adult bone marrow stem cells. There are already companies that will bank bone marrow, for example, http://www.cellslimited.com/bigcells.htm and http://www.stemcellbanking.com/. The reasoning behind this is that it is thought that stem cell numbers decline as you age, therefore you can harvest more when you are younger. Further, one of the advantages of obtaining adult rather than cord blood stem cells is their lifespan when frozen. Thus, most stem cell banks offer to store cord blood stem cells for a maximum of 25 years. This is acceptable if they are used to treat childhood leukemias or possibly an ageing parent, but for babies, the cells would not be there when they are 50 and have a heart attack. If a human subject stored adult bone marrow stem cells when they were 40, they would be available for that subject up to the age of 65. There is one company that already offers to store G-CSF mobilised stem cells http://www.stemlife.com/international/stemlife_services/adult_collection.html. These cells could also be used for sports injuries and chronic wounds or for treating any other condition where tissue repair and regeneration would be beneficial. At present, injured racehorses are treated with MSCs by taking bone marrow, cultivating it ex vivo and then injecting the stem cells directly back into the site of injury (http://www.vetcell.com/index.htm and WO 2004/022078).

In a further embodiment, the harvested or cultivated cells may be used in tissue engineering, such as to produce bone or cartilage (see, for example, the methods described in Fishman et al (Expert Opin Biol Ther 11(12): 1623-35) and Hollander et al (Stem Cells 28(11): 1992-6).

In yet a further embodiment, the harvested or cultivated cells may targeted to damaged tissue in a recipient subject, for example to tissue damaged by any one or more of ischaemia, stroke, myocardial infarction, radiotherapy, chemotherapy, auto-immune disease or physical injury. This may be achieved by introducing genes encoding specific receptors, adhesion molecules or antibodies into the cultivated cells. Alternative methods include attaching antibodies to the surface of the cells which antibodies can target the damaged tissue, or fusing the cells with cells that express surface moieties (eg chemokine receptors or adhesion molecules) that target the damaged tissue. Methods for targeting cells in this way are known in the art.

As described above, both HSPCs and MSCs have therapeutic potential in the treatment of a wide variety of diseases and conditions, and so it will be appreciated that HSPCs and/or MSCs harvested according to the methods of the invention may be useful to treat various diseases. Accordingly, a seventh aspect of the invention provides a method for repairing damaged blood vessels, for tissue regeneration, for treating myocardial infarction, stroke, heart disease, peripheral ischaemia, for treating diabetes, autoimmune disease (eg liver cirrhosis, rheumatoid arthritis, scierederma, graft versus host disease, rejection of organ allografts), systemic lupus erythematosus, multiple sclerosis, cystic fibrosis or other respiratory disease, for immunosuppression, for treating physical injuries (eg sports injuries) or for the healing of chronic wounds, the method comprising the step of administering a population of HSPCs and/or MSCs that has been harvested from a subject according to any of the methods of the first to sixth aspects of the invention described above (eg after administration of a beta adrenergic receptor (AR) agonist and optionally an inhibitor of the CXCR4/CXCL12 chemokine axis to a subject), and optionally administering a further therapeutic agent, to a subject; optionally wherein the beta AR agonist is a selective beta AR agonist.

This aspect of the invention includes a population of HSPCs and/or MSCs that has been harvested from a subject according to any of the methods of the first to sixth aspects of the invention described above (eg after administration of a beta adrenergic receptor (AR) agonist and optionally an inhibitor of the CXCR4/CXCL12 chemokine axis to a subject), and optionally a further therapeutic agent, for use in repairing damaged blood vessels, tissue regeneration, treating myocardial infarction, treating stroke, treating heart disease, treating peripheral ischaemia, treating diabetes, treating autoimmune disease (eg liver cirrhosis, rheumatoid arthritis, sclerederma, graft versus host disease, rejection of organ allografts), treating systemic lupus erythematosus, treating multiple sclerosis, treating cystic fibrosis or other respiratory disease, treating immunosuppression, treating physical injuries (eg sports injuries) or in the healing of chronic wounds; optionally wherein the beta AR agonist is a selective beta AR agonist.

Similarly, this aspect of the invention also includes a population of HSPCs and/or MSCs that has been harvested from a subject according to any of the methods of the first to sixth aspects of the invention described above (eg after administration of a beta adrenergic receptor (AR) agonist and optionally an inhibitor of the CXCR4/CXCL12 chemokine axis to a subject), and optionally a further therapeutic agent, in the manufacture of a medicament for repairing damaged blood vessels, tissue regeneration, treating myocardial infarction, treating stroke, treating heart disease, treating peripheral ischaemia, treating diabetes, treating autoimmune disease (eg liver cirrhosis, rheumatoid arthritis, sclerederma, graft versus host disease, rejection of organ allografts), treating systemic lupus erythematosus, treating multiple sclerosis, treating cystic fibrosis or other respiratory disease, treating immunosuppression, treating physical injuries (eg sports injuries) or for the healing of chronic wounds; optionally wherein the beta AR agonist is a selective beta AR agonist (eg a selective beta-3 AR agonist).

In an embodiment, the further therapeutic agent is an agent that is known to be suitable to treat any one or more of repairing damaged blood vessels, tissue regeneration, myocardial infarction, stroke, heart disease, peripheral ischaemia, diabetes, autoimmune disease (eg rheumatoid arthritis, sclerederma, graft versus host disease, rejection of organ allografts, multiple sclerosis), systemic lupus erythematosus, multiple sclerosis, cystic fibrosis or other respiratory disease, immunosuppression, physical injuries (eg sports injuries) or healing of chronic wounds.

It will be appreciated that the subject to which the harvested cells is administered may be the same or different subject from which the cells were harvested.

Terms such as “treating” or “treatment” or “to treat” refer to both therapeutic measures that cure, slow down, lessen symptoms of, and/or halt progression of a diagnosed pathologic condition or disorder and prophylactic or preventative measures that prevent and/or slow the development of a targeted pathologic condition or disorder.

As well as the clinical use of cells that have been harvested from a subject, it will be understood that the mere mobilisation of HSPCs and/or MSCs within a subject (eg caused by administration of the beta AR agonist and an inhibitor of the CXCR4/CXCL12 chemokine axis) may also have clinical benefit. For example, once HSPCs and/or MSCs are mobilised into the peripheral blood, it is expected that they will have the capacity to naturally home to sites of tissue damage, inflammation or disease so as to stimulate repair without the need to remove the cells from the subject first. Hence, an eighth aspect of the invention provides a beta AR agonist and an inhibitor of the CXCR4/CXCL12 chemokine axis, and optionally a further therapeutic agent, for use in repairing damaged blood vessels, tissue regeneration, treating myocardial infarction, treating stroke, treating heart disease, treating peripheral ischaemia, treating diabetes, treating autoimmune disease (eg liver cirrhosis, rheumatoid arthritis, sclerederma, graft versus host disease, rejection of organ allografts), treating systemic lupus erythematosus, treating multiple sclerosis, treating cystic fibrosis or other respiratory disease, treating immunosuppression, treating physical injuries (eg sports injuries) or for the healing of chronic wounds, optionally wherein the beta AR agonist is a selective beta-3 AR agonist.

This aspect of the invention includes the use of a beta AR agonist and an inhibitor of the CXCR4/CXCL12 chemokine axis, and optionally a further therapeutic agent, in the manufacture of a medicament for repairing damaged blood vessels, tissue regeneration, treating myocardial infarction, treating stroke, treating heart disease, treating peripheral ischaemia, treating diabetes, treating autoimmune disease (eg liver cirrhosis, rheumatoid arthritis, sclerederma, graft versus host disease, rejection of organ allografts), treating systemic lupus erythematosus, treating multiple sclerosis, treating cystic fibrosis or other respiratory disease, treating immunosuppression, treating physical injuries (eg sports injuries) or for the healing of chronic wounds, optionally wherein the beta AR agonist is a selective beta-3 AR agonist.

Similarly, this aspect of the invention also includes a method for repairing damaged blood vessels, for tissue regeneration, for treating myocardial infarction, stroke, heart disease, peripheral ischaemia, for treating diabetes, autoimmune disease (eg liver cirrhosis, rheumatoid arthritis, sclerederma, graft versus host disease, rejection of organ allografts), systemic lupus erythematosus, multiple sclerosis, cystic fibrosis or other respiratory disease, for immunosuppression, for treating physical injuries (eg sports injuries) or for the healing of chronic wounds, the method comprising the step of administering a beta AR agonist and an inhibitor of the CXCR4/CXCL12 chemokine axis, and optionally a further therapeutic agent, to a subject, optionally wherein the beta AR agonist is a selective beta-3 AR agonist.

The further therapeutic agent may be an agent that is suitable to treat the disease or condition as outlined above in relation to the sixth aspect of the invention.

A ninth aspect of the invention provides a population of HSPCs and/or MSCs isolated from a subject, wherein the subject has been administered a beta AR agonist and optionally an inhibitor of the CXCR4/CXCL12 chemokine axis, optionally wherein the beta AR agonist is a selective beta-3 AR agonist. In relation to isolating MSCs from a subject, it will be appreciated that it is not essential to administer the inhibitor of the CXCR4/CXCL12 chemokine axis when the MSCs are isolated directly from bone marrow since mobilisation of the cells is not necessary in this instance. However, in relation to isolating HSPCs, it is necessary to administer both a beta AR agonist and an inhibitor of the CXCR4/CXCL12 chemokine axis so as to mobilise the cells so that they can be harvested. Similarly, when MSCs are isolated from the peripheral blood, it is necessary to administer both a beta AR agonist and an inhibitor of the CXCR4/CXCL12 chemokine axis so as to mobilise the cells.

A tenth aspect of the invention provides a kit of parts comprising (i) a selective beta-3 AR agonist and (ii) an inhibitor of the CXCR4/CXCL12 chemokine axis. Preferences for (i) and (ii) include any of those described above in relation to earlier aspects of the invention. It is particularly preferred if the kit of parts comprises (i) a selective beta-3 AR agonist (eg BRL37344) and (ii) a CXCR4 antagonist (eg AMD-3100).

It is appreciated that the kit will be useful in providing the necessary agents to carry out the earlier aspects of the invention, such as to mobilise and harvest HSPCs and/or MSCs from a subject, or to treat various diseases or conditions by mobilising HSPCs and/or MSCs within a subject. In this case, it may be desirable to store the cells for later use and so the kit may further comprise a suitable agent to freeze the cells, such as liquid nitrogen.

Preferably, the kit of parts is for use in medicine.

In the context of the invention, it will be understood that the beta AR agonist (eg selective beta-3 AR agonist) and inhibitor of the CXCR4/CXCL12 chemokine axis (eg CXCR4 antagonist) are generally administered in admixture with a suitable pharmaceutical excipient diluent or carrier selected with regard to the intended route of administration and standard pharmaceutical practice. The agents will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. The formulations may take the form of tablets, capsules, ovules, elixirs, solutions or suspensions, which may contain flavouring or colouring agents, for immediate-, delayed- or controlled-release applications. Depending upon the disorder and subject to be treated, as well as the route of administration, the agents may be administered at varying doses. Preferably, the formulation is a unit dosage containing a daily dose or unit, daily sub-dose or an appropriate fraction thereof, of the agent or active ingredient.

An eleventh aspect of the invention provides a method for stimulating proliferation of MSCs, the method comprising contacting the MSCs with a beta AR agonist, optionally wherein the beta AR agonist is a selective beta-3 AR agonist. The method may further comprise administering an inhibitor of the CXCR4/CXCL12 chemokine axis. Preferences for the respective administrations include those defined above.

By stimulating proliferation we include the meaning of increasing the number of MSC cells in a population of cells that comprises MSCs. Thus, the methods may be used to expand the number of MSCs in an existing population of MSCs, or the methods may be used to enrich a population of mixed cells for MSCs. For example, the methods may be used to increase the number and/or proportion of MSCs in a mixed cellular sample, such as a sample derived from bone marrow or adipose tissue.

It will be appreciated that the methods of the eleventh aspect of the invention may be performed in vivo or in vitro.

In one embodiment, the method is performed in vitro and the cells are cultivated in vitro. Suitable culture methods include those described above. When the method is performed in vitro, the beta AR agonist and optionally the inhibitor of the CXCR4/CXCL12 chemokine axis, is preferably administered to the cellular sample either immediately after harvesting (eg from bone marrow or adipose tissue) or within the first week of culturing.

The beta AR agonist may be a general beta AR agonist including any of those described above such as isoproterenol, epinephrine and norepinephrine. Preferably, however, the beta AR agonist is a selective beta-3 AR agonist such as BRL37344, Mirabegron or CL316243, or any of the other beta AR agonists described herein, for example in relation to the first or second aspects of the invention.

In any aspect of the invention for mobilising and/or harvesting HSPCs and/or MSCs, or stimulating proliferation thereof, the method or use may further comprise administering a COX inhibitor (eg Naproxen, Aspirin) and/or a Free Fatty Acid Hydrolyse (FFAH) inhibitor (eg URB597) to the subject. Thus, it will be appreciated that the above kits of parts may further comprise a COX inhibitor (eg Naproxen, Aspirin) and/or a Free Fatty Acid Hydrolyse (FFAH) inhibitor (eg URB597).

The invention includes a method of identifying an inhibitor of the CXCR4/CXCL12 chemokine axis in a subject, wherein the method comprises the steps of: (i) administering a beta AR agonist to the subject; (ii) administering a test agent to the subject; and (iii) assessing the amount of HSPCs and/or MSCs in a sample from the subject. If the amount of HSPCs and/or MSCs in the sample from the subject is increased relative to the amount of HSPCs and/or MSCs in the sample from the subject when the test agent is not administered, or to a reference amount (ie a baseline level of HSPCs and/or MSCs in a sample from the subject), then the test agent is expected to be an inhibitor of the CXCR4/CXCL12 chemokine axis. In an embodiment, the method further comprises testing the ability of the test agent to inhibit the CXCR4/CXCL12 chemokine axis. Preferences for the administration regimes and routes are as described above. It is envisioned that the sample is a blood sample but it may be any biological sample such as lymph or any other bodily fluid.

The invention includes a method of identifying an agonist of the beta AR receptor (eg a selective beta-3 AR agonist) in a subject, wherein the method comprises the steps of: (i) administering a test agent to the subject (ii) administering an inhibitor of the CXCR4/CXCL12 chemokine axis to the subject; and (iii) assessing the amount of HSPCs and/or MSCs in a sample from the subject. If the amount of HSPCs and/or MSCs in the sample from the subject is increased relative to the amount of HSPCs and/or MSCs in the sample from the subject when the test agent is not administered, or to a reference amount (ie a baseline level of HSPCs and/or MSCs in a sample from the subject), then the test agent is expected to be an agonist of the beta AR receptor (eg a selective beta-3 AR agonist). In an embodiment, the method further comprises testing the ability of the test agent to agonise a beta AR receptor (eg a beta-3 AR receptor). Preferably, the test agent is administered chronically to the subject, for example at regular intervals or continuously over a long period of time, for example over at least 1 hour, 2 hours, 0.5 days, 1 day, 2 days, 3 days, 4 days and 5 days, and preferably over at least 3-5 days such as over at least 4 days. It is also preferred if the inhibitor of the CXCR4/CXCL12 chemokine axis is administered acutely to the subject. Most preferably, the test agent is administered chronically to the subject over a period of at least 1 hour, 2 hours or 0.5 days (eg at least 1 day, 2 days, 3 days, 4 days, or 5 days) before the inhibitor of the CXCR4/CXCL12 chemokine axis. It is envisioned that the sample is a blood sample but it may be any biological sample such as lymph or any other bodily fluid. It will be appreciated that this method can be used to identify which beta AR agonists are most effective with respect to mobilising of HSPCs and/or MSCs.

In the current invention, the subject may be any mammal. It is intended that the subject is a human but it may also be a mouse, a rat or another rodent, a goat, a sheep, a horse, a cow, a rabbit, a dog, cat or other domestic pet, an ape or a monkey. The subject may be a racing or sporting animal, for example a racehorse, polo pony or greyhound.

The invention will now be described in more detail by reference to the following Figures and Examples.

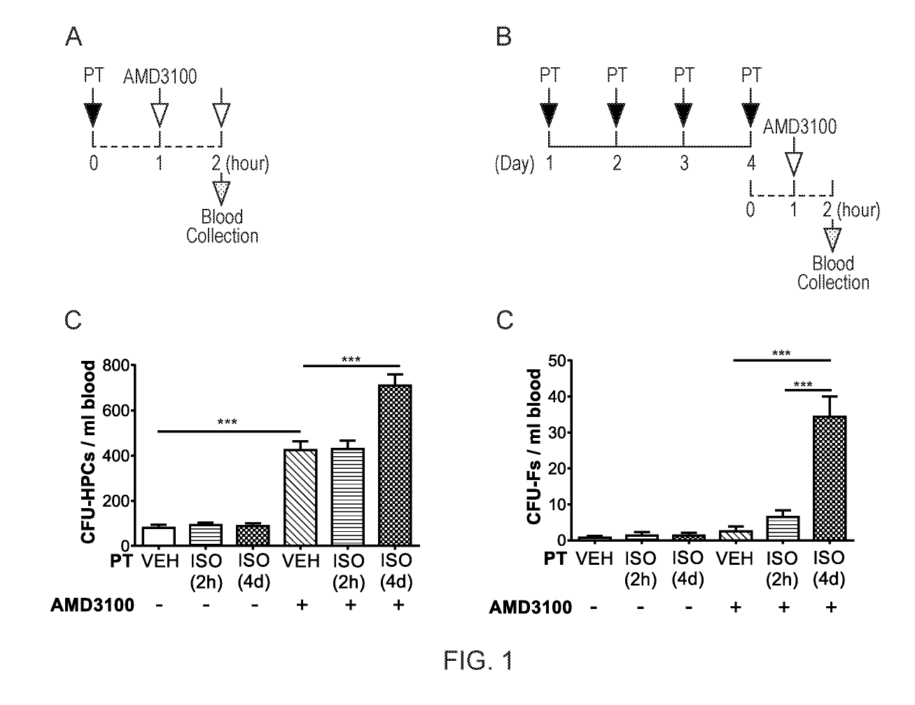

FIG. 1. βAR Activation Primes Progenitor Cells for Mobilization

(A) Experimental design for 2 hours (2 h) of pretreatment (PT). Mice were pretreated with isoproterenol (ISO) or vehicle (VEH) followed 1 hour later by the administration of AMD3100 or VEH. (B) Experimental design for 4 days (4 d) of PT. Mice were pretreated once daily for 4 days with ISO or VEH. 1 hour after the last injection, mice were administered AMD3100 or VEH. In both treatment regimens, 1 hour after AMD3100 or VEH injection blood was collected for analysis of circulating (C) CFU-HPCs and (D) CFU-Fs. CFU-HPCs and CFU-Fs are shown as colonies per ml of blood. n=8 mice per group. Data represented as mean±SEM. ***p<0.001 (one-way ANOVA).

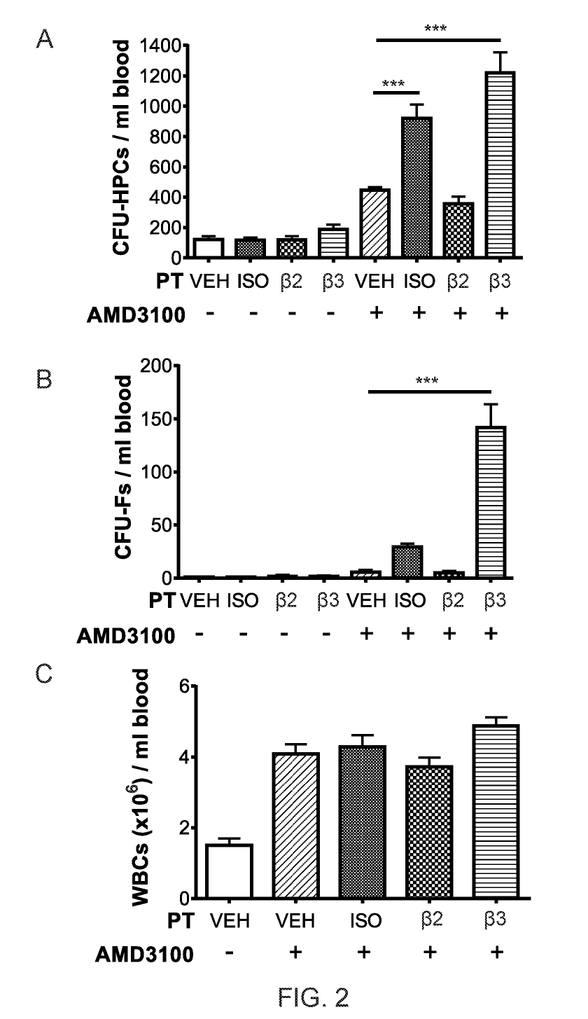

FIG. 2. β3AR Activation Primes Progenitor Cells for Mobilization

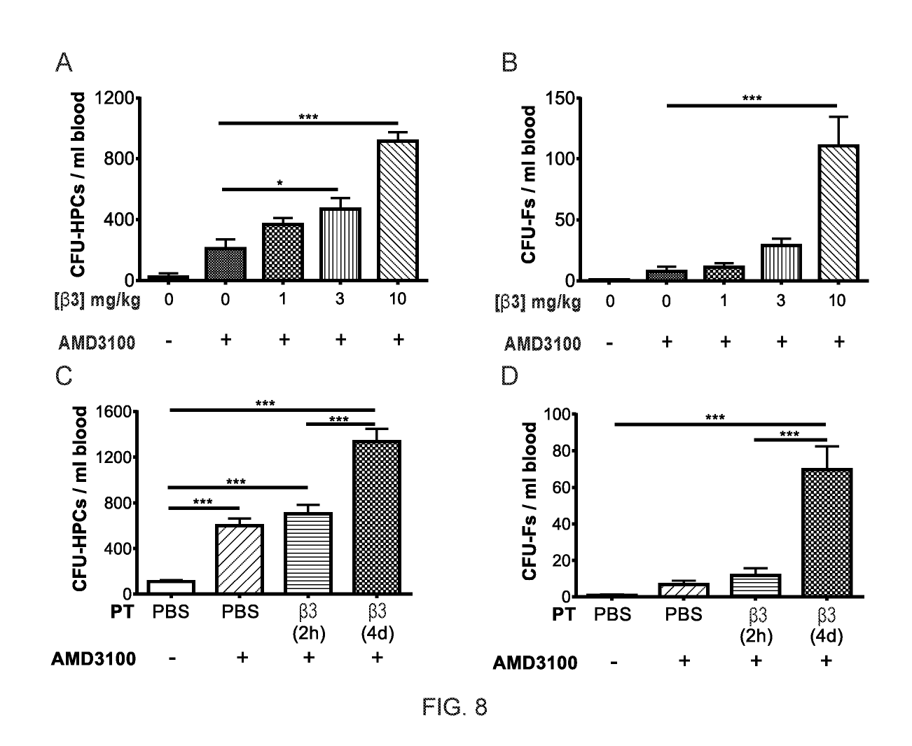

Mice were pretreated (PT) with isoproterenol (ISO), clenbuterol (β2), BRL 37344 (β3) or vehicle (VEH) once daily for 4 days. 1 hour after the last injection, mice were administered AMD3100 or VEH and 1 hour later blood was collected for analysis of circulating (A) CFU-HPCs, (B) CFU-Fs and (C) total white blood cells (WBCs). CFU-HPCs and CFU-Fs are shown as colonies per ml of blood. n=4-12 mice per group. Data represented as mean±SEM. ***p<0.001 (one-way ANOVA). See also FIG. 8.

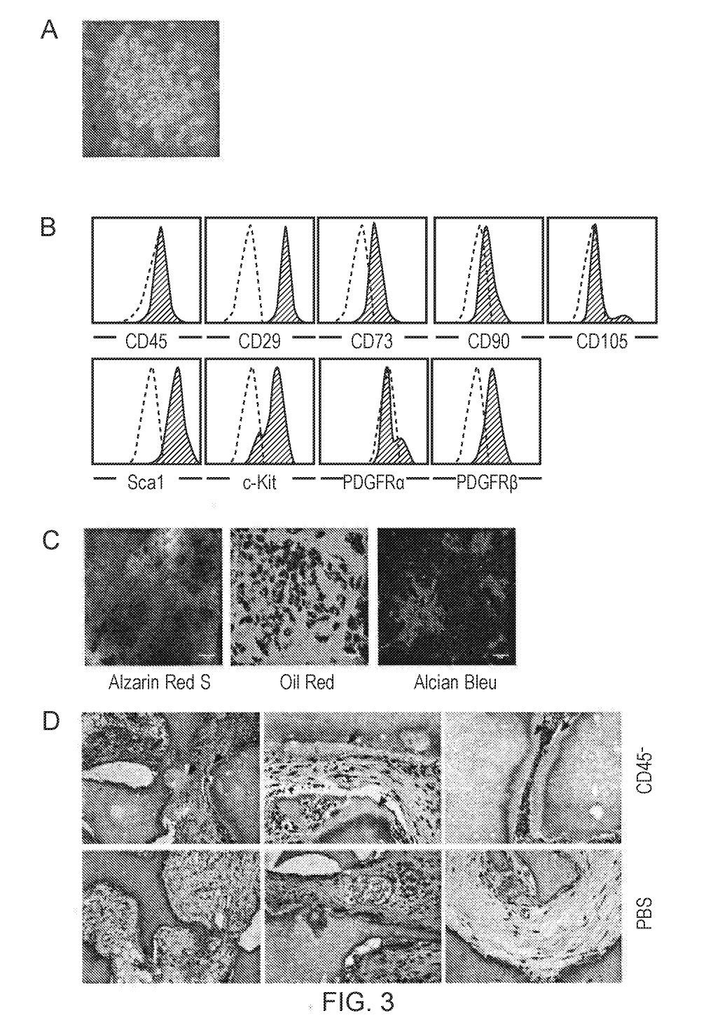

FIG. 3. Characterization of Mobilized CFU-Fs

Mice were pretreated with BRL 37344 (β3) once daily for 4 days. 1 hour after the last injection, mice were administered AMD3100 and 1 hour later blood was collected for analysis of circulating CFU-Fs. (A) Panel shows light microscopy of a representative CFU-F. (B) Histograms of surface marker expression on culture-expanded CFU-F cells as determined by flow cytometry. Shaded histograms represent marker expression and open dashed-line histograms represent fluorescence minus one (FMO) controls. (C) Panel shows representative trilineage differentiation staining of FACS sorted CD45⁻ culture-expanded CFU-Fs into osteocytes (Alizarin Red S), adipocytes (Oil Red) and chondrocytes (Alcian Bleu). (D) Histological analysis (H/E staining) showing In vivo bone formation (black arrows) of transplanted CD45− population with hydroxyapatite/tricalcium phosphate implants in mice.

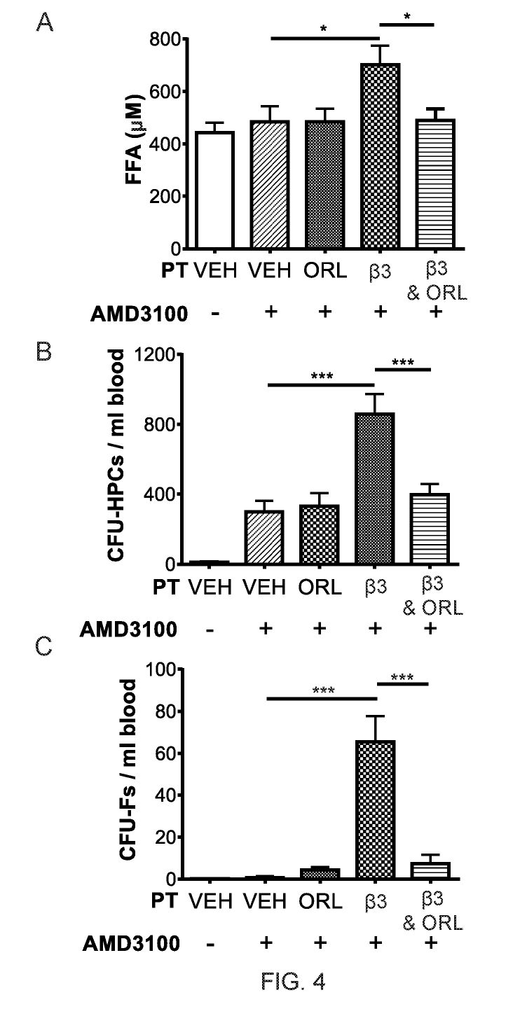

FIG. 4. Inhibition of Lipolysis Abrogates the Mobilization of Progenitor Cells in Response to β3-AR Activation

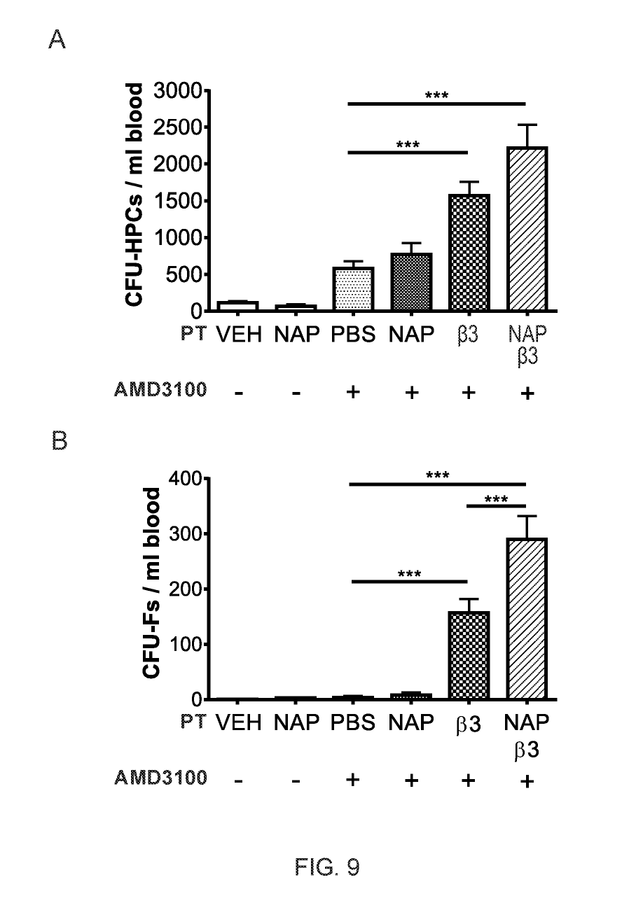

Mice were pretreated (PT) with BRL 37344 (β3) or vehicle (VEH) in the presence or absence of orlistat (ORL) (FIG. 1B) once daily for 4 days. 1 hour after the last injection, mice were administered AMD3100 or VEH and 1 hour later blood was collected for analysis of circulating (A) free fatty acids (FFAs) (B) CFU-HPCs and (C) CFU-Fs. FFAs are shown as μM and CFU-HPCs and CFU-Fs are shown as colonies per ml of blood. n=4-12 mice per group. Data represented as mean±SEM. *p<0.05, **p<0.01, ***p<0.001 (one-way ANOVA). See also FIG. 9.

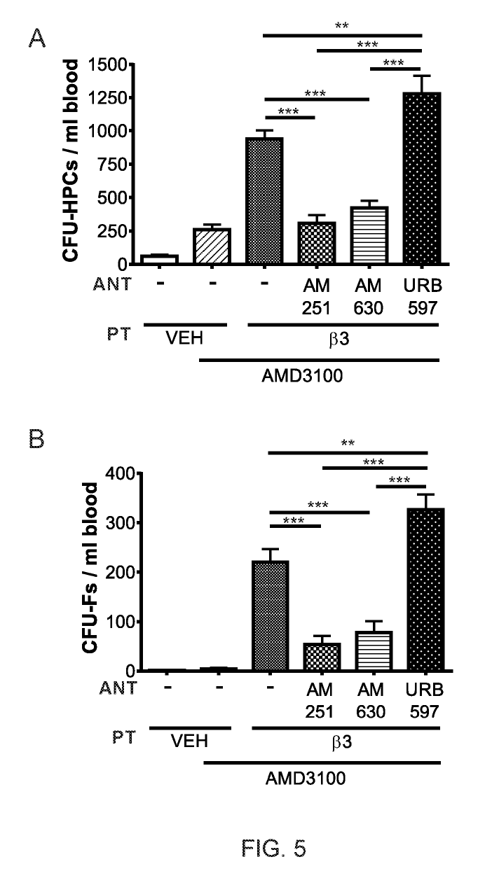

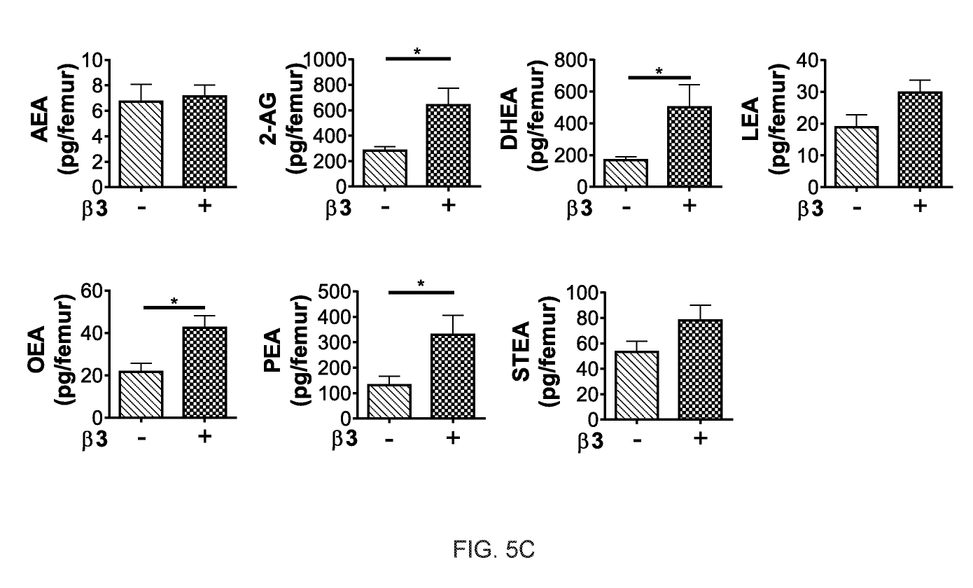

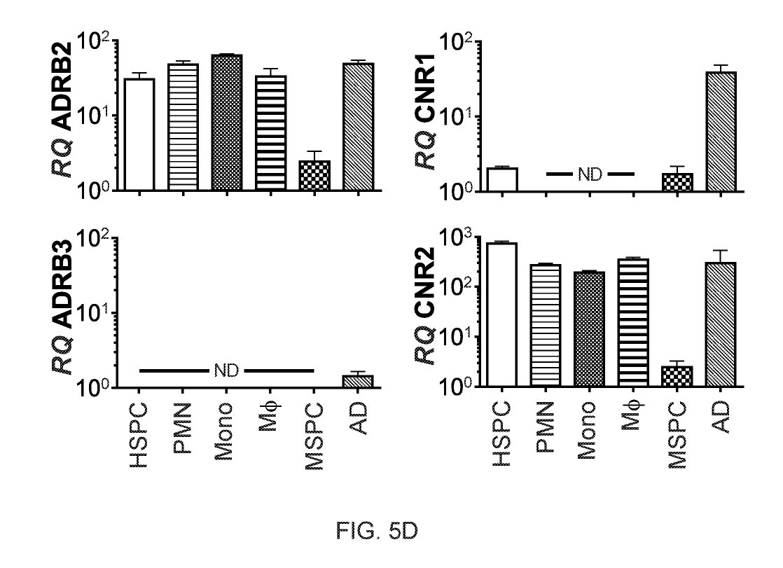

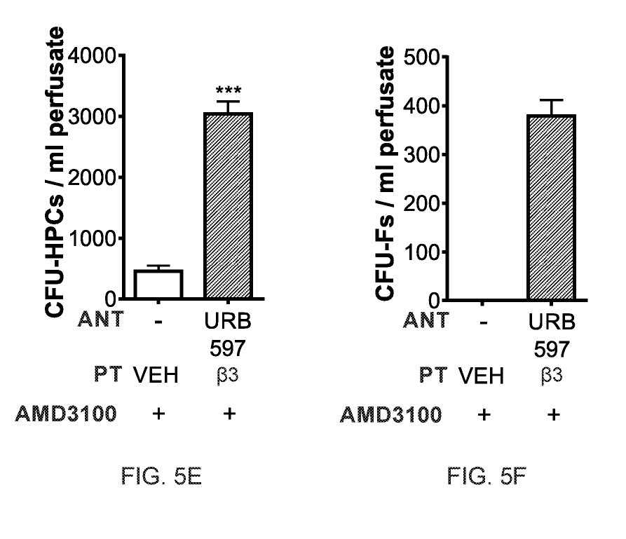

FIG. 5. β3-AR agonists Stimulate Generation of Lipid Mediators in the Bone Marrow that Prime Progenitor cells via Cannabinoid Receptors

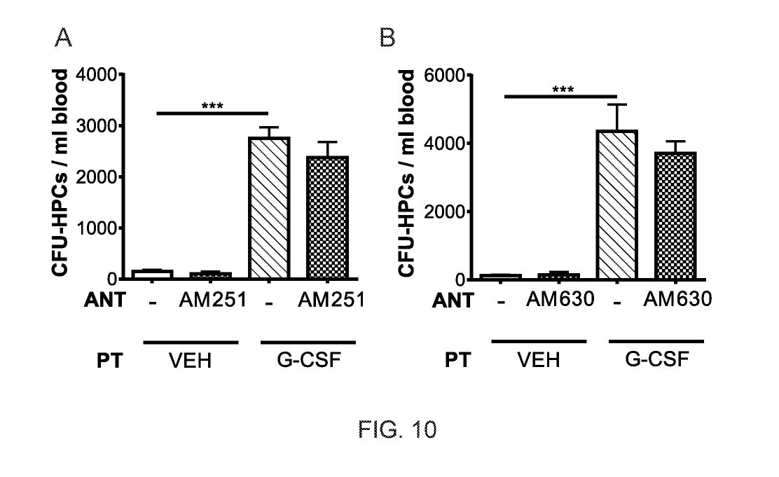

Mice were pretreated (PT) with BRL 37344 (β3) or vehicle (VEH) in the presence or absence of AM251, AM630 CB1 and CB2 antagonists (ANT) respectively or URB597 a FAAH inhibitor as indicated once daily for 4 days. 1 hour after the last injection, mice were administered AMD3100 or VEH and 1 hour later blood was collected for analysis of circulating (A) CFU-HPCs and (B) CFU-Fs. CFU-HPCs and CFU-Fs are shown as colonies per ml of blood. n=8-20 mice per group. In the next experiments, mice were pretreated with URB597 in the presence or absence of BRL 37344 once daily for 4 days. 2 hours after the last injection, bone marrow was collected for (C) lipid mediator quantification by UPLC/ESI-MS/MSC. Representative graphs shown (n=18-21 with bone marrow of 3 mice pooled together). (D) Real-time qRT-PCR of relative mRNA expression of ADRB2, ADRB3, CNR1 and CNR2 in bone marrow HSPCs, neutrophils (PMN), monocytes (Mono), macrophages (Mφ), MSPCs and subcutaneous adipose tissue (AD). Data are shown relative to the lowermost expressing sample for each gene. ND=Not detectable. (E-F) Mice were pretreated with URB 597, BRL 37344 or VEH once daily for 4 days as indicated. 1 hour after the last injection, mice underwent perfusion of the right hind limb. The hind limb was infused with AMD3100 or VEH for 10 minutes and further perfused for 50 minutes while collecting the perfusate for analysis of (E) CFU-HPCs and (F) CFU-Fs. CFU-HPCs and CFU-Fs are shown per ml perfusate (n=4-6). Data represented as mean±SEM. (A,B) *p<0.05, **p<0.01, ***p<0.001 (one-way ANOVA). (C,E,F)***p<0.001 (unpaired student t-test). See also FIG. 10.

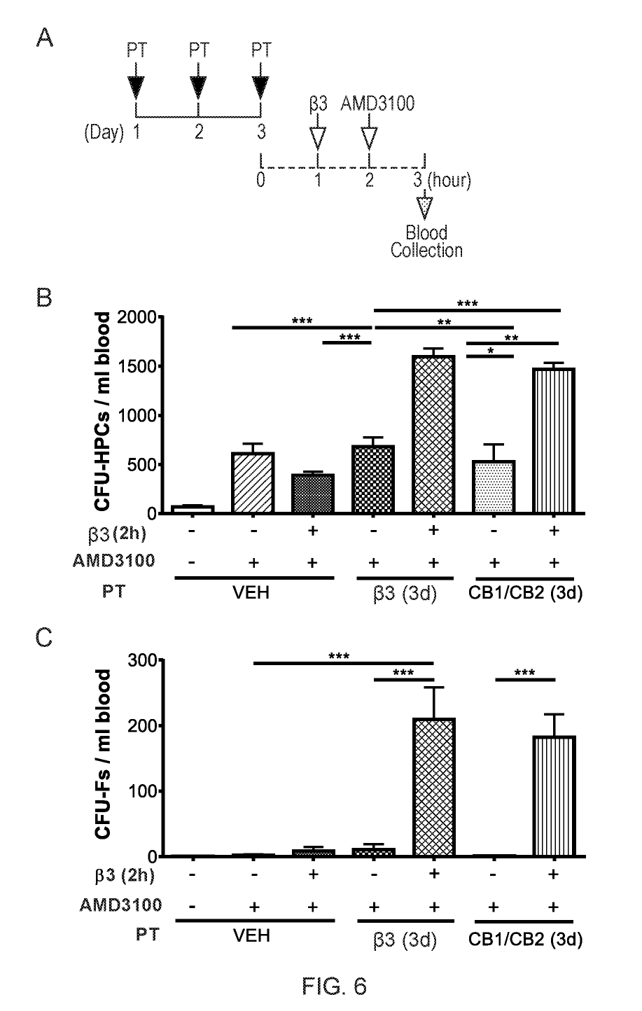

FIG. 6. Cannabinoid Receptor Signalling is Required but not Sufficient for Priming Progenitor Cells for MobilizationExperimental design (A). Mice were pretreated (PT) with URB597, ACEA (CB1), GP1a (CB2), BRL 37344 (β3) or vehicle (VEH) once daily on 3 consecutive days (3 d). On the fourth day, mice were either treated with BRL 37344 (β3) or VEH. 1 hour after last injection, mice were administered AMD3100 or VEH, and 60 minutes later blood was collected via cardiac puncture for enumeration of circulating CFU-HPCs (A) and CFU-Fs (B). CFU-HPCs and CFU-Fs are shown per ml of blood (n=6-10 mice per group). Data represented as mean±SEM. *p<0.05, **p<0.01, ***p<0.001 (one-way ANOVA).

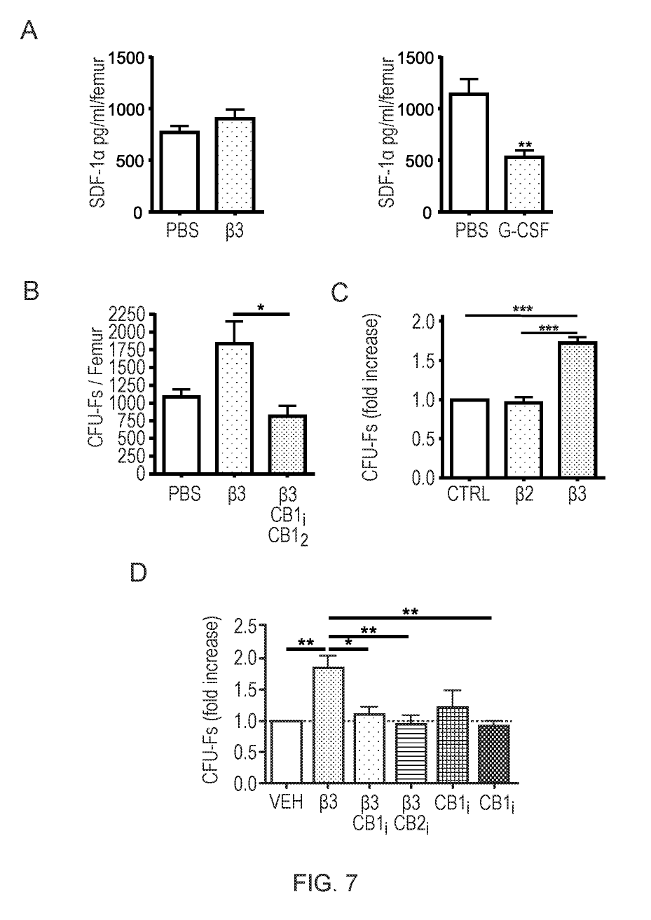

FIG. 7. β3-AR Activation and Cannabinoid Signalling Induces an Increase in Bone Marrow-derived CFU-Fs in vitro and in vivo.

(A) Mice were pretreated with BRL 37344 (β3), G-CSF or vehicle (VEH) once daily on 4 consecutive days. 2 hours after the final BRL 37344 injection or 24 hours after the final G-CSF injection, bone marrow was collected for quantification of CXCL12 levels (n=16 and n=6-8, respectively). (B) Bone marrow cells were collected from naïve mice and were treated in vitro with clenbuterol (β2), BRL 37344 (β3) or vehicle (VEH). CFU-Fs per 10⁶ cells are shown (n=6 mice per group). (C) Mice were pre-treated with AM 251, AM 630, BRL 37344 (β3) or vehicle (VEH) once daily on 4 consecutive days. 2 hours after the last injection, bone marrow cells were collected for enumeration of CFU-Fs. CFU-Fs per 10⁶ cells are shown (n=6-8 mice per group). (D) CB₁ receptor antagonist AM 251 (CB1ₗ, CB₂ receptor antagonist AM 630 (CB2ₗ, BRL 37344 (β3) or vehicle. Data represented as mean±SEM. (A) **p<0.01 (unpaired student t-test) and (B-D) *p<0.05, **p<0.01, ***p<0.001 (one-way ANOVA).

FIG. 8. P3AR Activation Regulates the Mobilization of Progenitor cells; dose-response and cel kinetics

Mice were pretreated once daily for 4 days with BRL 37344 (β3) (0-10 mg/ml as indicated) or vehicle (VEH). One hour after the last injection, mice were administered AMD3100 or VEH and one hour later blood was collected for analysis of circulating (A) CFU-HPCs and (B) CFU-Fs. Mice were pretreated either one hour (2 h) or once daily for 4 days (4 d) with BRL 37344 (β3) or vehicle (VEH). One hour after the last injection, mice were administered AMD3100 or VEH and one hour later blood was collected for analysis of circulating (C) CFU-HPCs and (D) CFU-Fs. (A-D) CFU-HPCs and CFU-Fs are shown as colonies per ml blood. (A-B) n=3-8 mice per group. (C-D) n=12 mice per group. Data are means±SEM. *p<0.05, **p<0.01, ***p<0.001 (one-way ANOVA). See also FIG. 2.

FIG. 9. Inhibition of Cyclooxygenase (COX) Enzyme Enhances Mobilization of Progenitor cells by β3AR Activation

Mice were pretreated with Naproxen (NAP), BRL 37344 (β3) or vehicle (VEH) once daily for 4 days. One hour after the last injection, mice were administered AMD3100 or VEH and one hour later blood was collected for analysis of circulating (A) CFU-HPCs and (B) CFU-Fs. CFU-HPCs and CFU-Fs are shown as colonies per ml blood. n=4-8 mice per group. Data are means±SEM. ***p<0.001 (one-way ANOVA). See also FIG. 4.

FIG. 10. G-CSF-induced Mobilization of Haematopoietic progenitor cells is Independent of Cannabinoid Receptor Signalling

Mice were pretreated with G-CSF or vehicle in the presence or absence of (A) AM251 or (B) AM630 once daily on 4 consecutive days. Twenty-four hours after last injection, blood was collected for analysis of circulating CFU-HPCs. CFU-HPCs are shown as colonies per ml blood. n=4-8 mice per group. Data are means±SEM. ***p<0.001 (one-way ANOVA).

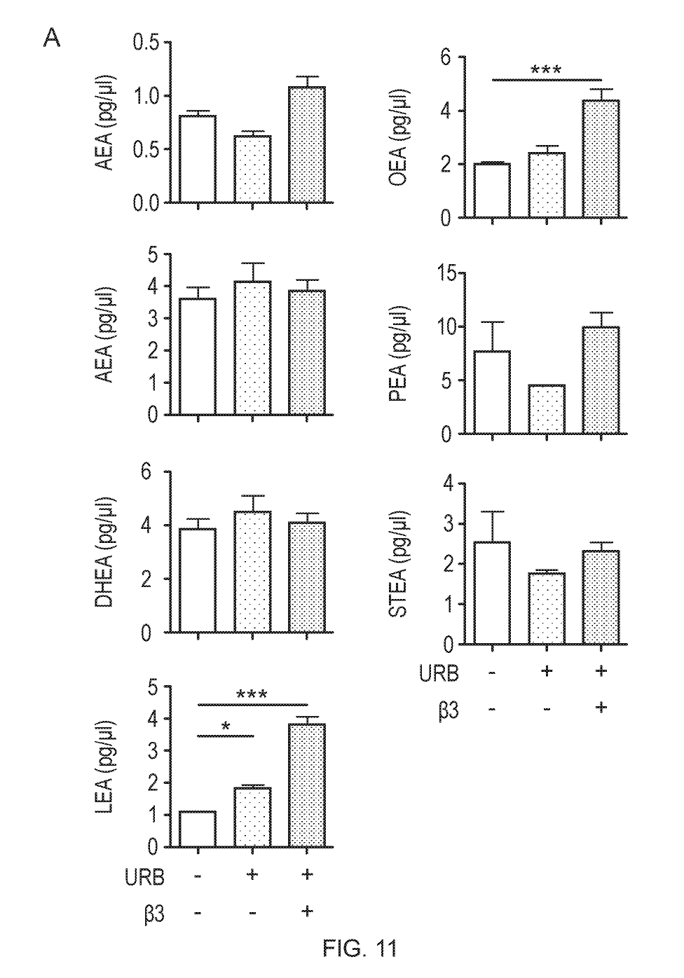

FIG. 11. β₃-adrenoceptor activation induces upregulation of lipid mediators. (A) Mice were pretreated with FAAH inhibitor URB 597 (URB; 0.5 mg/kg) or vehicle PBS v/v 2.5% EthOH prior treatment with BRL 37344 (β₃; 10 mg/kg i.p.) or dH₂O once daily on 4 consecutive days. Two hours after last injection, blood was collected via cardiac puncture and the levels of lipid mediators in blood plasma were quantified by mass spectrometry. (n=18-21; plasma of three mice pooled together). Data are displayed as mean±SEM. *p<0.05, ***p<0.001 (one-way ANOVA). See also FIG. 5.

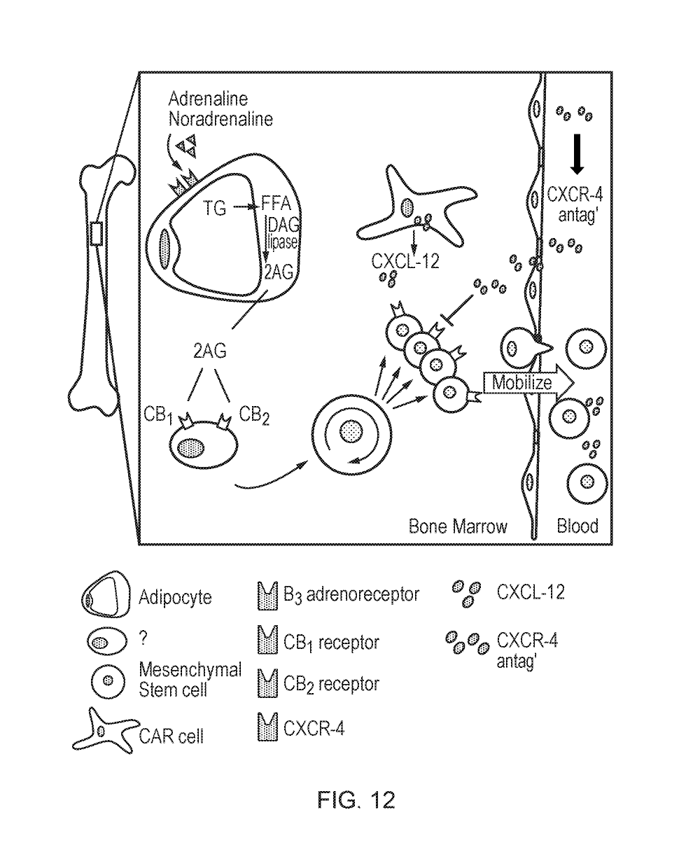

FIG. 12. Illustration summarising the ability of beta-3 agonists to stimulate generation of lipid mediators locally in the bone marrow, that regulate stem cell mobilisation.

FIG. 13. Amino acid sequence of human beta-3 AR (SEQ ID No: 1).

FIG. 14. Amino acid sequence of human CXCL12 (SEQ ID No: 2).

FIG. 15. Amino acid sequence of human CXCR4 (SEQ ID No: 3).

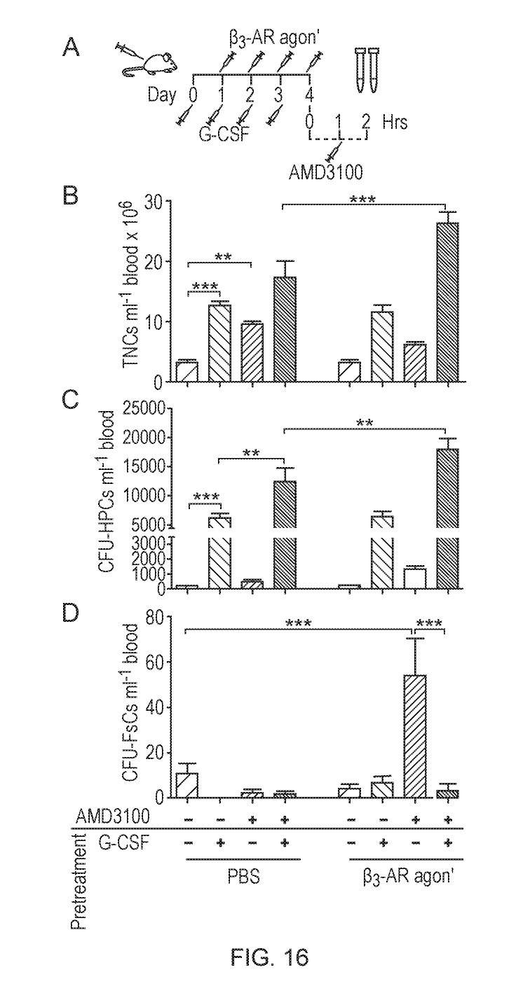

FIG. 16. G-CSF pretreatment impairs β3-AR agon′/AMD3100 mobilisation of MSCPs.

Experimental design (A). BALB/c mice were pretreated with G-CSF (100 μg/kg i.p.) or vehicle (PBS) once daily for 4 consecutive days. One day post first G-CSF injection, mice were pretreated with β₃-AR agonist (10 mg/kg i.p.) or vehicle (dH₂O) once daily on 4 consecutive days. One hour after last injection, mice were administered AMD3100 (5 mg/kg i.p.) or vehicle (PBS), and an hour later blood was collected via cardiac puncture for enumeration of circulating TNCs (B), HSPCs (C) and MSPCs (D). TNCs, CFU-HPCs and CFU-Fs per ml blood (n=4-9). Data of two independent experiments (N=2), displayed as meant SEM. *p<0.05, **p<0.01 (one-way ANOVA; Bonferroni's Multiple Comparison Test).

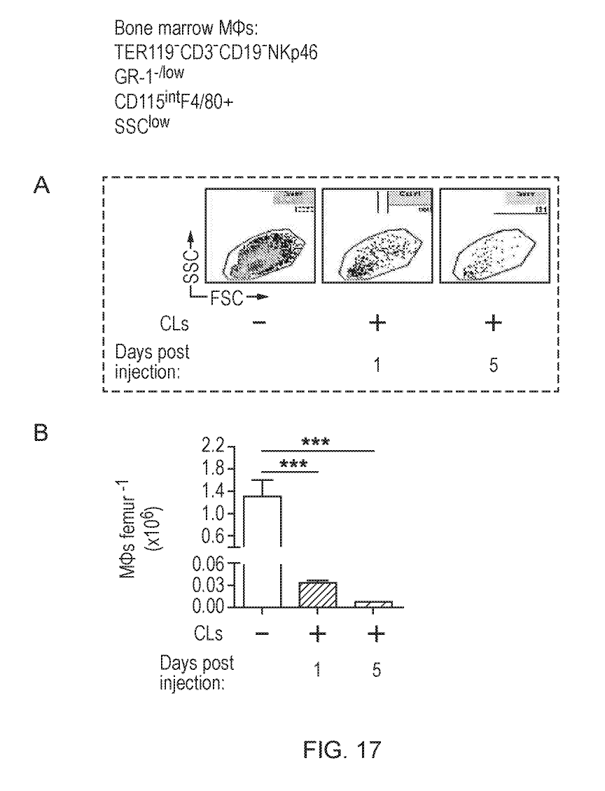

FIG. 17. Mice were treated with clodronate on day 0 and CD115+, F4/80+ macrophages were quantified in the bone marrow by flow cytometry on day 1 and 5. (A) representative flow cytometry plots. (B) absolute numbers of macrophages.

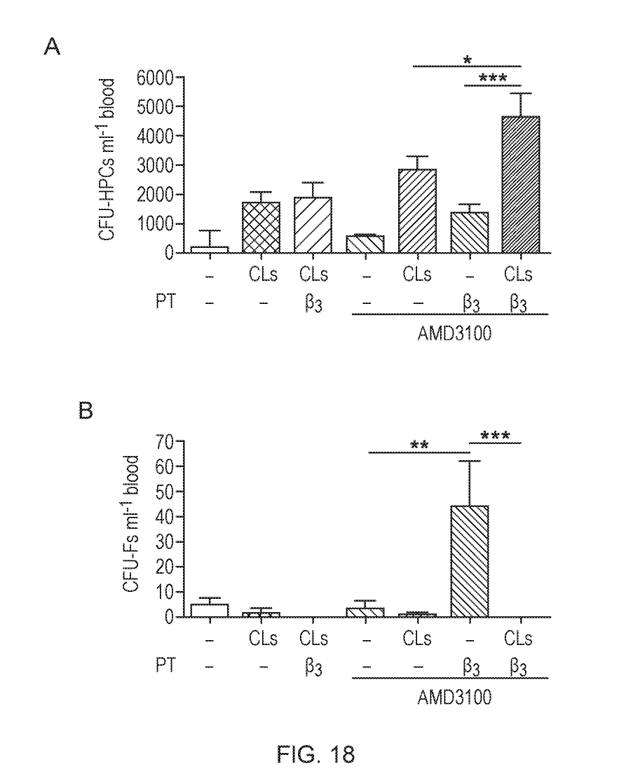

FIG. 18. Mice were treated with a single injection of clodronate (CL) or vehicle control (−) on day 0. Mice were subsequently treated with β3 Adrenergic agonist (β3) or vehicle control (−) daily on days 1-4. On day 4 some mice received a single subcutaneous injection of AMD3100 as indicated. Mice were killed 1 hour after administration of AMD3100 or vehicle control. Circulating numbers of HPCs (A) and CFU-Fs (B) were determined using specific colony forming assays.

### EXAMPLE 1

### Beta 3 AR Aqonists Stimulate Localised Production of Endocannabinoids by Bone Marrow Adipocytes and Prime Haematopoietic and Mesenchymal Stem Cells for Mobilization

Summary

Treatment of mice with beta 3 adrenergic agonists over 4 days was shown to prime both haematopoietic and mesenchymal stem cells for mobilization in response to a CXCR4 antagonist. Investigation of the underlying mechanism revealed that the effect was due to the localised production lipid mediators within the bone marrow. Thus mass spectrometry revealed significantly elevated levels of AG, DHEA and PEA following in vivo administration of the b3 agonist and inhibition of the enzymes that stimulated the production (Lipase) or degradation (FAAH) of these mediators inversely affected the priming response. Further the priming effect of the beta 3 adrenergic agonists was inhibited in part, by CBI and CB2 receptor antagonists. The function of bone marrow adipocytes has hitherto been an enigma and these results suggest that they may provide a local source of lipid mediators that regulate stem cell activity in the bone marrow.

Introduction

The function of adipocytes in the bone marrow is an enigma and commonly these cells are considered merely as space fillers, their numbers increasing when the bone marrow is destroyed by irradiation or when haematopoiesis declines with age. Based on the results presented herein we propose a new paradigm whereby activation of beta 3 adrenoreceptors on bone marrow adipocytes promotes lipolysis and the localised production of lipid mediators (ethanolamides/endocannabinoids) that act locally to prime both HSPCs and MSCs for mobilization into the blood. This suggests a new function for bone marrow adipocytes as a source of short acting lipid mediators that exert their effects locally in the bone marrow environment to regulate stem cell activity.

Results

Activation of β3ARs Primes HSPCs and MSPCs for Mobilization by a CXCR4 Antagonist

We sought to investigate whether β adrenergic receptor agonists could stimulate the mobilization of MSCs into the blood. We examined both acute (2 h) and 4 day treatments, and included combined treatment regimens in our analyses (FIGS. 1A and 1B). Consistent with our previous work while treatment with the CXCR4 antagonist, AMD3100, stimulated mobilization of HSPCs into the blood, no colony forming units-fibroblasts (CFU-Fs; a measure of MSCs) were detectable. Acute treatment (2 h) of mice with the general β adrenoreceptor agonist, isoproterenol, alone or in combination with acute administration of the CXCR4 antagonist did not enhance the mobilization of HSPCs above AMD3100 alone and did not result in detectable numbers of CFU-Fs in the blood (FIGS. 1C and 1D). A 4 day pre-treatment with the isoproterenol alone did not change circulating numbers of HSPCs or CFU-Fs. However, when AMD3100 was administered to mice pre-treated for 4 days with isoproterenol, HSPC numbers were significantly elevated over treatment with the CXCR4 antagonist alone (FIG. 1C) and, uniquely, CFU-Fs were mobilized (FIG. 1D). Thus treatment with a general beta adrenergic agonist over 4 days appears to prime both HSPCs and MSCs for mobilization by the CXCR4 antagonist.

Activation of β3ARs is Important to Prime Stem Cells for Mobilization

To de-lineate the specific beta adrenoreceptor subtype involved in this mobilization response, we performed the same experiments, including the beta 2 and beta 3-specific adrenergic agonists Clenbuterol and BRL37344, respectively. While the beta 2 adrenoreceptor-specific agonist did not stimulate HSPC or MSC mobilization, when administered alone or in combination with the CXCR4 antagonist, the b3 adrenoreceptor specific agonist significantly boosted circulating numbers of HSPCs and CFU-Fs as compared to that observed with isoproterenol and AMD3100 in combination (FIGS. 2A and 2B). These data suggest that activation of beta 3 adrenoreceptors primes stem cells for mobilization by the CXCR4 antagonist. In contrast, mobilization of total white blood cells that is significantly increased with the CXCR4 antagonist alone was not further elevated by the adrenergic agonists (FIG. 2C).

Further experiments showed a dose response relationship for mobilization of HSPCs and CFU-Fs in response to the CXCR4 antagonist when mice were pre-treated with 0-30 mg/ml BRL37344 (FIG. 8A) and confirmed that the effect of the beta 3 agonist, like the general agonist, required the pre-treatment of mice over 4 days to see this effect (FIG. 9B). Similarly to the experiments using the general βAR agonist, the effect of the β3AR-specific agonist was only observed after the mice were pretreated for 4 days (FIG. 9C and FIG. 9D).

CFU-Fs Mobilized into the Blood Exhibit Characteristics of MSPCs

Experiments were next performed to see whether the CFU-Fs mobilized into the blood following 4 day beta 3 pre-treatment and acute administration of the CXCR4 antagonist exhibited characteristics of MSCs. These blood-derived CFU-Fs were shown to be plastic-adherent cells that formed colonies when plated at low density (FIG. 3A). Following expansion in culture flow cytometry revealed that these cells expressed the classic markers of MSCs (CD29, CD90, CD73, CD105, Sca-1 and c-Kit) while being negative for CD45 (FIG. 3B). In vitro, these cells could be differentiated along osteogenic, chondrogenic and adipocyte lineages using standard protocols (FIG. 3C). Bone formation could be visualised in vivo (FIG. 3D). As such we consider that CFU-Fs measured in the blood represent MSPCs (Mesenchymal stem and progenitor cells).

Stem Cell Priming by β3AR Activation is Dependent on Lipolysis

Beta 3 adrenergic receptors are primarily expressed on adipocytes and are known to regulate lipolysis and thermogenesis. Thus in adipocytes, beta 3 adrenergic agonists stimulate lipase induced hydrolysis of triglycerides, leading to the generation of glycerol and free fatty acids (FFA) that are detectable in the plasma. Given that the bone marrow contains adipocytes we first sought to rule out the possibility that stem cell priming was associated with effects of beta 3 adrenergic agonists on lipolysis by pre-treating mice with Orlistat a general lipase inhibitor. Analysis of FFA levels in plasma showed an increase following treatment with the b3 adrenergic agonist over 4 days that was attenuated by pre-treatment of the mice with Orlistat (FIG. 4A). Moreover, HSPC and CFU-F mobilization induced by 4 day pre-treatment with the b3 adrenergic agonist was completely abrogated when mice were pre-treated with Orlistat (FIGS. 4B and 4C). These data suggested that stimulation of lipolysis mediated by Beta3 adrenergic agonist is necessary for stem cell priming.

Stem Cell Priming by β3AR Activation is Dependent on CB1 and CB2

Increased lipolysis may increase the availability of polyunsaturated fatty acid precursors, that are required to generate bioactive lipids (for example, eicosanoids and related oxylipins), as well as to increase the concentration of phospholipid-derived endocannabinoids and N-acyl ethanolamides (Ueda et al, 2013). We therefore sought to investigate whether these lipid signalling molecules were implicated in priming stem cells for mobilization. Use of a general COX inhibitor (Naproxen) ruled out a role for prostanoids in this process (FIG. 9). Indeed, inhibition of COX caused a significant increase in the mobilization of both HSPCs or CFU-Fs stimulated by b3 adrenergic agonist and CXCR4 antagonist in combination FIGS. 5A and 5B. In contrast, antagonists of either the cannabinoid receptor 1 (CB1-AM251) or cannabinoid receptor 2 (CB2-AM630) significantly suppressed Beta 3 adrenergic agonist mediated mobilization for both HSPCs and CFU-Fs in response to a CXCR4 antagonist, indicating a significant role for endocannabinoid signalling in this response (FIGS. 5A and 5B).

In general the effects of lipid mediators are limited both spatially and temporally by enzymes that efficiently degrade them. In the case of endocannabinoids fatty acid amide hydrolyse (FAAH) is key in their hydrolysis and inactivation (Hwang et al, 2010). We therefore examined whether inhibition of FAAH with a specific inhibitor (URB597) would affect stem cell mobilization in vivo. Our results show that mobilization of HSPCs and CFU-Fs by treatment with beta 3 agonist in combination with the CXCR4 antagonist was significantly enhanced when mice were pre-treated with URB597, consistent with a role of endocannabinoids in this response (FIGS. 5A and 5B).

β3AR-Specific Activation Stimulates Production of Endocannabinoids and N-acyl Ethanolamides in the Bone Marrow

Endocannabinoids are lipid mediators that are chemically unstable and rapidly degraded as such they are usually generated at their site of action. To examine whether endocannabinoids were generated locally in the bone marrow microenvironment, mass spectrometry was performed on mouse plasma and bone marrow samples following treatment with BRL37344 over 4 days. In these experiments, FAAH was inhibited in all mice to reduce the rapid hydrolysis of these lipid mediators. In mice in which only FAAH was inhibited, the endocannabinoids anandamide (AEA) and 2-arachidonoyl glycerol (2-AG), as well as several N-acyl ethanolamides (oleoyl ethanolamide, OEA, arachidonoyl ethanolamide AEA, palmitoyl ethanolamide PEA, Docosatetraenoyl ethanolamide, DHEA, linoleoyl ethanolamide LEA and stearoly ethanolamide STEA), could be detected in the bone marrow, with high basal levels of 2-AG, PEA and DHEA (FIG. 5C). Moreover, bone marrow levels of 2-AG, PEA and DHEA were significantly increased following β3AR-specific activation in combination with FAAH inhibition compared with that of mice in which only FAAH had been inhibited (FIG. 5C). To our knowledge this is the first demonstration that β3AR-specific agonists can stimulate the generation of endocannabinoids and their congeners locally in the bone marrow. Taken together, these results suggest that beta 3 agonists promote stem cell mobilization from the bone marrow due to the localized production of endocannabinoids/ethanolamides, some of which act via CB1 and CB2 receptors.

To give further insight into the cell types in the bone marrow that may be responding to the beta 3 adrenergic agonist or endocannabinoids, expression of beta 2, beta 3 adrenoreceptors and CB1 and CB2 receptors were examined by RT-PCR on purified populations of stem cells, leukocyte subsets and adipocytes (FIG. 5D). Consistent with previous reports (Bouaboula et al, 1993; Galiegue et al, 1995; Galve-Roperh et al, 2013; Li et al, 2010) we show expression of b2 adrenoreceptors on all of the cell types tested but selective expression of beta 3 adrenoreceptors on adipocytes. CBI receptors were expressed at high levels by adipocytes and low levels by both stem cell populations, while CB2 was expressed at high levels by all cell populations with the exception of MSCs.

HSPCs and MSPCs are Mobilized Directly from the Bone Marrow

To establish whether the observed changes in the number of progenitor cells circulating in the peripheral blood was due to increased egress directly out of the bone marrow, an in situ perfusion model of the murine hind limb was used. This model involves cannulation of the femoral artery and vein to allow the femur and tibia bone marrow to be perfused in (Pitchford et al, 2009; Pitchford et al, 2010) isolation. The CXCR4 antagonist is administered directly via infusion into the femoral artery over 10 mins, while cells that exit the bone marrow and enter the circulation are collected via cannulation of the femoral vein. This technique permits a more accurate quantification of mobilized bone marrow progenitors over a set time frame, as it prevents complications associated with mobilized cells trafficking into peripheral organs or homing back to the bone marrow. In mice pre-treated with vehicle and infused with the CXCR4 antagonist, HPCs but not MSCs were mobilized from the bone marrow (FIGS. 5E and 5F). Four day pretreatment with b3 adrenergic agonist in combination with the FAAH inhibitor significantly enhanced both HPC and MSC egress from the bone marrow in response to infusion of the CXCR4 antagonist (FIG. 5E). This result is consistent with the supposition that the bone marrow is the source of progenitors mobilized into the blood following this pharmacological treatment.

CB1 and CB2 are Required but Not Sufficient to Prime HSPCs and MSPCs for Mobilization

The actions of ethanolamides, in particular DHEA and PEA are not solely mediated via CB1 and CB2 receptors. To investigate whether stem cell priming for mobilization was mediated by these receptors we utilised ACEA and GP1a selective CB1 and CB2 agonists respectively Arachidonyl-2-chloroethylamide (ACEA) and CB2 (N-(Piperidin-1-yl)-1-(2,4-dichlorophenyl)-1,4-dihydro-6-methylindeno[1,2-c]pyrazole-3-carboxamide (GP1a). Initial experiments showed that a 4 day pre-treatment with ACAE and GP1a in combination was not sufficient to prime MSCs or HSPCs for mobilization even in the presence of the FAAH inhibitor to prevent hydrolysis of ACEA. As previously shown (FIGS. 1A and B), a 2-hour activation of β3AR, without additional activation of CB1 and CB2, did not increase the mobilization of HSPCs or MSPCs by CXCR4 antagonism (FIGS. 6B and C). However, when mice were pre-treated with ACAE and GP1a in combination for 3 days, stem cells were mobilized if the beta 3 adrenergic agonist was administered on the fourth day, prior to the CXCR4 antagonist (FIGS. 6A and 6B). A similar effect could be seen using a 3 day pre-treatment with either AHAE or GP1a alone, followed by administration of the beta 3 agonist and CXCR4 antagonist on day 4, but the level of mobilization was significantly less than that achieved using these agonists in combination. Taken together, FIG. 6 suggests that both CB1 and CB2 receptors are both required for optimal priming of stem cells. The requirement of beta 3 adrenergic agonist administration on day 4 after the 3 day pre-treatment with the CB1 and CB2 agonists suggests that final stage in stem cell priming requires lipid mediators that act on distinct receptors to CB1 and CB2 these lipid mediators are generated in response to treatment with the beta 3 adrenergic agonist on day 4. Overall, this data implies that the priming response is complex, involving multiple lipid mediators, receptors and mechanisms.

4 days of β3AR-Specific Activation Does Not Change Bone Marrow CXCL12 Levels but Does Increase CFU-F Numbers

A reduction in the level of CXCL12 in the bone marrow is associated with HSPC mobilization in response to G-CSF treatment. It has been reported that β3AR is expressed on bone marrow stromal cells and that its activation reduces CXCL12 production by these cells in the short term (Katayama et al, 2006; Mendez-Ferrer et al, 2008; Mendez-Ferrer et al, 2010). We show here that, in contrast to treatment with G-CSF, 4 days of β3AR-specific agonist treatment had no effect on levels of CXCL12 in the bone marrow, which suggests that HSPC and MSPC mobilization in response to β3AR-specific activation and CXCR4 antagonism is not dependent on a reduction in bone marrow CXCL12 levels (FIG. 7A).

CB2 agonists have previously been reported to increase MSPC proliferation in vitro but only in mixed cultures of bone marrow mononuclear cells (Scutt and Williamson, 2007). Consistent with this finding, we observed an increase in the number of CFU-Fs in bone marrow cultures following β3AR-specific but not β2AR-specific activation in vitro (FIG. 7B). Furthermore, β3AR-specific activation in vivo led to an increase in CFU-F numbers in the bone marrow. This was inhibited when mice were co-administered CB1 and CB2 antagonists (FIG. 7C). This data shows that β3AR-specific activation stimulates MSPCs in the bone marrow through generation of endocannabinoids.

Discussion

Taken together, our data suggest a novel function of BMAT as a localised source of lipid mediators that regulate stem cell activity. We have provided the first evidence that beta 3 agonists administered systematically drive the local generation of a unique profile of endocannabinoids/ethanolamides in the bone marrow that act to prime both HSPCs and MSCs for mobilization.

The physiological function of adipocytes in the bone marrow is still considered an enigma. BMATs are yellow as they contain an intermediate number of mitochondria as compared to high levels that are present in brown fat and low levels in white fat. In addition to their distinct phenotype they are unresponsive to high fat diets or anorexia, and there is no evidence to suggest that they play a role in the bodies general energy metabolism or thermogenesis. Indeed, BMAT is commonly considered a space filler in bone marrow expanding with age as haematopoiesis declines, or in response to irradiation-induced injury.

Beta 3 adrenoreceptors are expressed at high levels on adipocytes and beta 3 agonists are known to stimulate lipolysis in peripheral fat stores. BMAT similarly expresses b3 adrenoreceptors and contains high levels of triglycerides. In fact, infusion of the general β agonist, isoproterenol, directly into the vasculature of the canine femur was shown to lead to the release of FFAs into the femoral vein, suggesting that lipolysis could also be stimulated locally in the bone marrow (Tran et al, 1981). In our study, the fact that the lipase inhibitor, Orlistat, abrogated the ability of the b3 agonist to prime both HSPCs and MSCs for mobilization led us to investigate the potential role of lipid mediators in this response.

BMAT expresses all the biochemical machinery to synthesise and degrade ethanolamides, and it has previously been reported that extracts of bone marrow contain ethanolamides at similar levels to those reported in the brain, where they are known to exert physiological effects (Bab et al, 2011). Analysis of lipids extracted from the bone marrow of naïve mice revealed the presence of endocannabinoids and N-acyl ethanolamides, with high basal concentrations of AG, DHEA and PEA which were all significantly elevated following administration of a selective β3 agonist in vivo. In contrast, other N-acyl ethanolamides, including AEA were present at low levels in naïve mice and remained unchanged following administration of the b3 agonist. Therefore, we have provided the first direct evidence that systemic β3AR-specific activation drives the local generation of a unique profile of endocannabinoids and N-acyl ethanolamides in the bone marrow. This suggests that BMAT is important in generating bioactive lipid mediators.

Ethanolamides are not chemically stable entities and are rapidly degraded, as such they are thought to act locally close to their site of generation. FAAH is an enzyme expressed in BM and other tissues that degrades ethanolamides and thereby limits their action, both temporally and spatially. In this study, we observed enhanced stem cell mobilization in response to β3 agonist/CXCR4 antagonist treatment when FAAH was inhibited, consistent with the concept that degradation of lipid mediators limits their actions. We also observed an increase in β3-stimulated stem cell mobilization when cyclooxygenase was inhibited, consistent with previous reports showing that inhibition of COX is associated with an increased bioavailability of the substrate (arachadonic acid) for generation of ethanolamides.

Ethanolamides are known to act as full or partial agonists of the cannabinoid receptors 1 and 2 (Sugiura, 2009). In the periphery, CB1 receptors are primarily expressed on pre-synaptic sympathetic neurones and function to reduce noradrenaline release (Elefteriou, 2008; Elefteriou et al, 2014), while CB2 receptors are expressed on cells of the immune system and on osteoblasts (Bab et al, 2011). With respect to the bone marrow a number of studies have reported effects of CB1 and 2 agonists on bone density and stem cell activity, by a number of distinct mechanisms. Release of noradrenaline from sympathetic nerves activates beta 2 adrenoreceptors on osteoblasts, inhibiting their differentiation and reducing mineralisation, thereby leading to bone loss. CB1 agonists are physiological antagonists of this process as they act pre-synaptically to reduce noradrenaline release from sympathetic nerves thereby attenuating bone loss. CB2 agonists act directly to enhance bone density by stimulating osteoblast proliferation. Thus both CB1 and CB2 agonists increase bone density. In terms of stem cells, CB2 agonists have been shown to stimulate the proliferation of MSCs when they are initially cultured from the bone marrow, an effect that is lost in pure cultures of MSCs. We report here that β3 agonists stimulate an increase in CFU-Fs in the bone marrow, an effect that is abrogated by CB1 and 2 antagonists, suggesting that lipid mediators that act via cannabinoid receptors expand MSCs in the bone marrow. In contrast, while HSPCs are known to express CB1 and CB2 receptors we did not observe any changes in the number of HSPCs, suggesting that other mechanisms must regulate their mobilization via CB1/CB2. In this respect, it has been reported that 2AG stimulates the migration of HSPCs and thus could potentially stimulate their migration to the vascular niche for mobilization (Patinkin et al, 2008).

Not all the effects of ethanolamides are mediated via CB1/2 receptors. Specifically, DHEA, originally termed synaptamide, is an ethanolamide that stimulates synaptogenesis and synaptic connectivity in a CB^{1/2}-independent manner, suggesting that its effects are mediated by other distinct receptors (Kim and Spector, 2013). Thus, given that we have observed a significant elevation of an array of ethanolamides in response to β3 administration, it is likely that a number of distinct receptors, cell types and potentially multiple mechanisms are operating to prime both MSCs and HSPCs for mobilisation following the administration of a β3 agonist. Indeed, the fact that we could not replicate the effect of B3 pre-treatment using a combination of CB1 and CB2 agonists, points to this conclusion.

It has previously been reported that the SNS plays a critical role in regulating HSPC mobilization in response to circadian oscillations with beta 2 adrenoreceptors regulating the clock genes while stimulation of beta 3 adrenoreceptors on osteoblasts was reported to reduce their production of CXCL12. In this context, the cells responding are those that are directly innervated by the SNS and changes in CXCL12 are occurring within a matter of hours to regulate HSPC mobilization over the course of a day. In the experiments presented here, stem cell mobilization was not seen when beta agonists were administered acutely to naïve mice, while the effects of longer term treatment with beta agonists has not previously been examined.

A number of clinical trials were set up to investigate whether mobilizing HSPCS with G-CSF can promote tissue repair in the context of both MI and stroke. To date only very modest positive effects have been reported. Use of β3 agonists in combination with a CXCR4 antagonist may be more effective given that this pharmacological strategy mobilizes both HSPCS and MSCs, but also unlike G-CSF it does not induce granulopoiesis and an increase in inflammatory cells.

Experimental Procedures

Animals

Female BALB/c mice weighing 22-25 g were purchased from Harlan Laboratories. All studies were carried out under the United Kingdom's Animals (Scientific Procedures) Act of 1986 and local ethical approval from Imperial College London.

Administration of Drugs

Mice were administered the β-adrenergic agonists isoproterenol (10 mg/kg i.p.), clenbuterol (5 mg/kg i.p.) and BRL 37344 (10 mg/kg i.p.), the cannabinoid receptor agonists ACEA (10 mg/kg i.p.) and GP1a (5 mg/kg i.p.), or vehicle either on 4 consecutive days or 2 hours prior to the cull. 1 hour after the last injection, mice were administered the CXCR4 antagonist AMD3100 (5 mg/kg i.p.) or vehicle. 1 hour later their blood was collected via cardiac puncture for enumeration of circulating CFU-HPCs and CFU-Fs. Mice were administered murine G-CSF (100 μg/kg i.p.) or vehicle on 4 consecutive days. On day 5, blood was collected via cardiac puncture for enumeration of circulating HSPCs.

The antagonists or inhibitors AM251 (5 mg/kg i.p.), AM630 (5 mg/kg), orlistat (50 mg/kg i.p.), URB 597 (0.5 mg/kg), naproxen (10 mg/kg oral gavage) or vehicle, were administered 60 minutes prior to the injection of the mobilizing treatment, BRL 37344, ACEA, GP1a or G-CSF.

In Situ Perfusion of Mouse Hind Limb

Mice were administered BRL 37344 (10 mg/kg i.p.) or vehicle on 4 consecutive days. 1 hour after the last injection, the mice were anaesthetized. The femoral vein and artery were exposed and cannulated in situ immediately after the hind limb was isolated by occlusion of the surrounding arteries as previously described (Pitchford et al, 2009; Pitchford et al, 2010). Perfusion buffer was infused via the arterial cannula and removed from the venous cannula using a Minipuls Peristaltic Pump (Anachem). The hind limb was initially infused with AMD3100 (0.1 mM) or vehicle for 10 minutes using an infusion/withdrawal pump (Harvard Instruments) and was further perfused for 50 minutes with perfusion buffer (Pitchford et al, 2010). The perfusate was collected over 60 minutes and then centrifuged and resuspended in DMEM (Gibco) +20% fetal bovine serum for enumeration of CFU-HPCs and CFU-Fs.

CFU-HPC Assay

Harvested peripheral blood or perfusate was red blood cell-lysed and 1×10⁵ cells were added to tissue culture-treated petri dishes containing 1 ml of Methocult™ (M3434; StemCell Technologies) to select for CFU-HPCs. Cultures were incubated at 37° C. and quantified on day 12. Similarly, 1×10⁶ bone marrow cells harvested by bone marrow flush of the hind limb femur were plated for CFU-HPCs.

CFU-F Assay

Harvested peripheral blood or perfusate was red blood cell-lysed and 1×10⁶ cells were added to tissue culture-treated 6-well plates containing 3 ml of Mesencult™ (05502; StemCell Technologies) to select for CFU-Fs. Similarly, 1×10⁶ bone marrow cells harvested by bone marrow flush of the hind limb femur were plated for CFU-Fs. Cultures were incubated at 37° C. and media was changed on day 7. Bone marrow-derived CFU-Fs were enumerated on day 13 and blood- and perfusate-derived CFU-Fs were enumerated on day 21. Blood derived-CFU-Fs were further expanded and assessed for mesenchymal lineage markers by flow cytometry and following CD45 depletion (flow cytometry sorting of CD45 negative for mesenchymal trilineage differentiation (Supplemental Experimental Procedures).

Flow Cytometry Analysis and Fluorescence-Activated Cell Sorting

Flow cytometry was used to determine the expression of MSC markers on blood CFU-Fs that had been expanded in culture for 6 weeks. Cells were stained with fluorochrome-conjugated monoclonal antibodies directed against CD45, CD29, CD73, CD90, CD105, Sca-1, and c-Kit, Cells were analysed using a Fortessa (Becton Dickson). Fluorescence-activated cell sorting was performed on blood CFU-Fs expanded in culture for 6 weeks before performing mesenchymal trilineage differentiation. Cells were stained with fluorochrome-conjugated monoclonal antibodies directed against CD45. Cells were sorted using a FACS Aria II (Becton Dickson).

CXCL12 Enzyme-Linked Immunosorbent Assay

CXCL12 content in blood plasma and bone marrow supernatant was quantified using CXCL12 capture (MAB350) and detection (BAF351) antibodies (RnD systems), and the assay was performed according to the manufacturers' instructions. Recombinant mouse CXCL12 (RnD systems) was used to generate a standard curve.

Free Fatty Acid Assay

Free fatty acid content in blood plasma was quantified using a serum/plasma fatty acid detection Kit (Zen Bio) and the assay was performed according to the manufacturers' instructions. Data are expressed as μM concentration of free fatty acids.

Real-Time Quantitative PCR

Total RNA was isolated from cells or tissue using QIAamp RNA Mini Kit according to the manufacturers' instructions (Qiagen). Subsequent cDNA was prepared for each sample using a High Capacity cDNA Reverse Transcription Kit with RNase Inhibitor (Applied Biosystems) according to the manufacturers' instructions. qRT-PCR reactions were performed with Power SYBR® Green PCR Master Mix (Applied Biosystems), user-designed primers (Primer-BLAST-assisted; Invitrogen) and run on ViiA™ 7 Real-Time PCR System (Invitrogen). Data are shown relative to the lowermost expressing sample for each gene. Gene expression was normalized to the internal standard gene ACTB. Primer information is presented in the Supplemental Experimental Procedures.

Mass Spectrum and Chromatographic Analysis of Endocannabinoids and Congeners

Plasma and bone marrow flush (pool of 3 mice, n=1) were processed in order for the extraction of endocannabinoids and congeners (Astarita and Piomelli, 2009; Kingsley and Marnett, 2009). Samples were analysed on an electrospray (ESI) tandem quadrupole Xevo TQ-S mass spectrometer (MS/MS; Waters) coupled to an Acquity Ultrahigh Pressure Chromatography (UPLC) system, and the concentration of analytes was calculated (TargetLynx). Further details concerning extraction and analysis can be found in the Supplemental Experimental Procedures.

Materials

The source, dose and vehicle for all drugs is shown in Table 1. Methocult and Mesencult proliferation kit (StemCell Technologies). Murine G-CSF (PeproTech). SDF-1α/CXCL12 ELISA (R&D Systems). Serum/Plasma Fatty Acid Detection Kit (ZenBio). Commercially available standards for A-EA, A-EA-d8, palmitoylethanolamine (P-EA), docosahexaenoyl (DH-EA), a-linleoyl ethanolamine (AL-EA), oleoyl ethanolamine (O-EA), stearoyl ethanolamine (ST-EA), linoleoyl ethanolamine (L-EA), 2-arachidonylglycerol (2-AG), 2-AG-d8 were purchased from Cayman Chemical Co (Ann Arbour, Mich., USA). Ultrapure water was tapped by a Purelab Flex system from Elga Process Water (Marlow, UK), Strata Si-1 (Silica) SPE tubes (Phenomenex, Macclesfield, UK). Chloroform and methanol (Chromosolv LC-MS grade—Sigma-Aldrich).

TABLE 1
In vivo reagents and solvents/vehicles

| Reagent | Company | Dose (in Vivo) | | Vehicle |
|---|---|---|---|---|
| Isoproterenol | Sigma Aldrich | 10 | mg/kg | dH₂0 |
| Clenbuterol | Sigma Aldrich | 5 | mg/kg | dH₂0 |
| BRL 37344 | Sigma Aldrich | 10 | mg/kg | dH₂0 |
| AMD3100 | Sigma Aldrich | 5 | mg/kg | PBS |
| Orlistat | Tocris Bioscience | 50 | mg/kg | PBS v/v 12.5% EthOH |
| AM251 | Tocris Bioscience | 5 | mg/kg | PBS v/v 6.25% EthOH |
| | | | | & 5% Tween 80 |
| AM630 | Tocris Bioscience | 5 | mg/kg | PBS v/v 6.25% DMF |
| | | | | & 5% Tween 80 |
| URB597 | Sigma Aldrich | 0.5 | mg/kg | PBS v/v 2.5% EthOH |
| Naproxen | Sigma Aldrich | 10 | mg/kg | PBS v/v 2% DMSO, |
| | | | | 5% Tween 80, 5% |
| | | | | castor oil & w/v 1% |
| GP1a | Tocris Bioscience | 5 | mg/kg | PBS v/v 2.8% EthOH |
| | | | | & 5% Tween 80 |
| ACEA | Enzo Life | 10 | mg/kg | PBS v/v 5% EthOH & |
| | Sciences | | | 5% Tween 80 |
| G-CSF | PeproTech | 100 | μg/kg | PBS |

TABLE 2
For flow cytometry antibodies

| Antibody | Clone | Company |
|---|---|---|
| TER119-FITC | TER119 | eBioscience |
| CD29-PE | eBioHMb1-1 | eBioscience |
| CD31-FITC | 390 | Biolegend |
| CD45-FITC | 30-F11 | BD |
| CD73-PE | TY/11.8 | Biolegend |
| CD90.2-PE | 30-H12 | Biolegend |
| CD105-PE | M17/18 | eBioscience |
| Ly-6A-E/Sca1-PECy7 | D7 | BD |
| CD117/C-kit-PE | 2B8 | eBioscience |
| CD140α/PDGFRα-APC | APA5 | eBioscience |
| CD140β/PDGFRβ-PE | APB5 | eBioscience |

TABLE 3
Primers for qRT-PCR

| Gene | Primer | Sequence | SEQ ID No |
|---|---|---|---|
| ADRB2 | F | CTGGTTGGGCTACGTCAACT | 4 |
| | R | CTTCCTTGGGAGTCAACGCT | 5 |
| ADRB3 | F | GCAGGAGGAAGATGGAAACC | 6 |
| | R | CAGTTACTGGAGACACCCGC | 7 |
| CNR1 | F | CGTTGAGCCTGGCCTAATCA | 8 |
| | R | GAACCAACGGGGAGTTGTCT | 9 |
| CNR2 | F | TTCGCCCACGCTTAGTGATT | 10 |
| | R | AGCTGGTGCAGGAATTCACA | 11 |
| ACTB | F | CTGTCGAGTCGCGTCCACCC | 12 |
| | R | GCTTTGCACATGCCGGAGCC | 13 |

Mesenchymal Trilineage Differentiation

Blood mobilized CFU-F colonies were first expanded for 6 weeks and flow cytometry active cell sorting was used to isolate CD45 negative population. These cells were then cultured for 3 weeks in adipogenic, osteogenic and chondrogenic differentiation media. Adipogenic medium contained 0.5 μM dexamethasone, 0.5 μM isobutyl-methylxanthine and 50 μM indomethacin. Osteogenic medium contained 1 nM dexamethasone, 20 μM β-glycerolphosphate and 50 mM ascorbic acid-2-phosphate. Serum-free chondrogenic medium contained 10 ng/mL TGF-61, 100nM dexamethasone, 50 μg/mL ascorbic acid-2-phosphate, 100 μg/mL sodium pyruvate, 40 μg/mL L-proline, 1× ITS+3 (Sigma-Aldrich) and 1.25 mg/mL BSA. Cells were then stained with Oil Red O, Alizarin Red S and Alcian Blue for adipocytes, osteoblasts and chondrocytes, respectively.

In Vitro Pretreatment of Bone Marrow CFU-Fs with β-Adrenoceptor Agonists

Cells were harvested by bone marrow flush of the hind limb femur, and 1×10⁶ cells were added to tissue culture treated 6-well plates containing 3 ml of Mesencult™ (05502; StemCell Technologies) to select for CFU-Fs, and incubated at 37° C. After fifteen minutes the plate was treated with a single dose of clenbuterol (10 μM), BRL 37344 (10 μM) or vehicle. Media exchange was performed on day 7 and CFU-Fs were enumerated on day 13.

Isolation and Culture of Cells Used for Gene Expression Studies

Bone marrow cells were isolated via bone marrow flush of femur, tibia and iliac crest. To isolate bone marrow neutrophils, cells were carefully layered over a previously prepared Percoll gradient (52%, 64%, 72% percoll fractions; Sigma-Aldrich) and centrifuged for 30 mins (1500 g). The enriched neutrophil layer was collected and washed twice with media.

Neutrophils isolated had expected 85-90% purity. Bone marrow monocytes were isolated using the EasySep™ Mouse Monocyte Enrichment Kit (StemCell Technologies) according to the manufacturer's instructions. Bone marrow HSPCs were isolated using the EasySep™ Mouse Hematopoietic Progenitor Cell Enrichment Kit (StemCell Technologies) according to the manufacturer's instructions. Bone marrow MSPCs were isolated by plastic adherence followed by immunomagnetic cell isolation. Briefly, bone marrow cells were cultured on tissue culture treated plastic flasks in DMEM (Gibco) supplemented with 20% FBS (Gibco), 5 ng/ml basic Fibroblast Growth Factor (bFGF; Peprotech), 2 U/ml heparin (Sigma-Aldrich) and 1% penicillin/streptomycin (Sigma-Aldrich). Cultured MSCs were enriched at passage 1 using EasySep™ Mouse Mesenchymal Stem/Progenitor Cell Enrichment Kit (StemCell Technologies) according to the manufacturer's instructions. Bone marrow macrophages were isolated by culture on plastic petri dishes in RPMI w L-Glutamine (Gibco) supplemented with 10% FBS, 100 ng/ml human M-CSF (PeproTech), 50 μM β-mercaptoethanol (Sigma-Aldrich) and 1% penicillin/streptomycin (Sigma-Aldrich). Adipose tissue was isolated from mouse inguinal fat pads.

Extraction of Endocannabinoids and N-acyl Ethanolamides from Mouse Plasma and Bone Marrow Flush

Bone marrow flushed from the femurs of 3 mice were pooled (6 ml in total) per treatment to give an n=1 sample. This volume was required to achieve sufficient concentrations of endocannabinoids and N-acyl ethanolamides for detection by UPLC/ESI-MS/MS. Extraction of lipids was carried out by addition of chloroform:methanol (2:1, v/v) (Astarita and Piomelli, 2009; Kingsley and Mamett, 2009). Specifically, ice cold chloroform:methanol was added to each bone marrow supernatant (ml per pooled bone marrow sample), followed by the internal standards AEA-d8 (20 ng) and 2AG-d8 (40 ng). The resulting suspensions were kept on ice for 30 min with occasional vortexing and centrifuged at 5000 rpm for 8 min, to separate the organic and aqueous phases. The organic layer (bottom) from each sample was then removed into a clean vial. The supernatant was evaporated under a fine stream of nitrogen. Once dried the extract was reconstituted in 1 ml of chloroform and semi-purified by SPE (Guo et al, 2010). Briefly, the silica cartridge was equilibrated with 5×1 ml cholorform, the extract was applied, the cartridge was washed with 2×1 ml chloroform and the endocannabinoids eluted with 5×1 ml chloroform:methanol (2:1 v/v). The remaining residue, reconstituted in 100 μL ethanol was stored at −20° C., for no more than 1 week, awaiting UPLC/ESI-MS/MS analysis.

UPLC/ESI-MS/MS Analysis

All analysis was performed on an electrospray (ESI) tandem quadrupole Xevo TQ-S mass spectrometer (Waters) coupled to an Acquity Ultrahigh Pressure Chromatography (UPLC) system. The system was controlled by MassLynx v4.1 Software. TargetLynx was used to construct calibration lines and calculate the concentration of analytes of interest. Optimised ESI-MS/MS conditions were achieved through the use of Intellistart within MassLynx software. Individual standards (100 pg/μl) were introduced into the spectrometer by direct infusion via the Xevo TQ-S integrated syringe pump (flow rate 10 μl/min) combined with UPLC solvent flow (rate 0.2 mL/min). All analytes were monitored on the positive ionisation mode. Capillary voltage was set at 2000V, source temperature at 150° C., desolvation temperature at 400° C. and the cone voltage at 20 V.

Chromatographic analysis of the endocannabinoids and congeners was performed on an Acquity UPLC® BEH Phenyl C18 column (1.7 μm, 2.1×50 mm) maintained at 25° C. supported with Acquity UPLC® BEH Phenyl VanGuard pre-column (1.7 μm, 2.1×5 mm). Sample injections were performed with the Acquity sample manager (Waters); the sample chamber temperature was set at 8° C. The injection volume was 3 μL and the flow rate 0.6 ml/min. Analytes were separated using an acetonitrile-based gradient system comprising two solvents: solvent A: water/glacial acetic acid 99.5:0.5 (v/v); solvent B: acetonitrile/glacial acetic acid 99.5:0.5 (v/v). The following gradient was used: Initial conditions 22% solvent B increasing linearly to 28% solvent B at 3min; 3.0-3.1 increase of solvent B to 55% and remaining at 55% up to 10.9 min; 10.9-11.0 min increase of solvent B to 80% and remaining at 80% up to 12.5 min; 12.5-12.51 decrease of solvent B to 22%.

In Vivo Bone Formation

Culture expanded CFU-F colonies were FACS sorted for the CD45 negative population. These cells were then seeded on hydroxyapatite/tricalcium phosphate (HA/TCP) granules overnight at 37° C. They were then transplanted in mice for six weeks. The implants were removed, fixed, gently decalcified and finally paraffin embedded for histological analysis by H/E staining as is standard procedure in the art.

### EXAMPLE 2

### Bone Marrow Macrophages Required for Mobilisation of Mesenchymal Progenitor Cells in Response to β3AR Agonist in Combination with AMD3100

To further investigate the mechanisms regulating the mobilisation of Haematopietic progenitor cells (HPCs) and mesenchymal progenitor cells (MPCs) mice were pretreated with clodronate liposomes which were shown to significantly reduce numbers of macrophages in the bone marrow (FIG. 1). Mice that were treated with clodronate were subsequently treated with β3 adrenergic agonist over 4 days followed by the CXCR4 antagonist AMD3100 on day 4. The data indicate that depletion of macrophages with clodronate significantly enhanced the mobilisation of HPCs with AMD3100 alone and with the β3AR agonist in combination with AMD3100. From these data we propose that bone marrow macrophages negatively regulate the mobilisation of HPCs from the bone marrow. In contrast the mobilisation of MPCs (as measured by CFU-Fs) was completely abrogated when macrophages were depleted from the bone marrow. These data indicate that bone marrow macrophages are required for the mobilisation of mesenchymal progenitor cells in response to β3AR agonist in combination with AMD3100.

### REFERENCES

Astarita, G., and Piomelli, D. (2009). Lipidomic analysis of endocannabinoid metabolism in biological samples. J. Chromatogr. B. Analyt Technol. Biomed. Life. Sci. 877, 2755-2767.

Bab, I., Smoum, R., Bradshaw, H., and Mechoulam, R. (2011). Skeletal lipidomics: regulation of bone metabolism by fatty acid amide family. Br. J. Pharmacol. 163, 1441-1446.

Bianco, P., Cao, X., Frenette, P. S., Mao, J. J., Robey, P. G., Simmons, P. J., and Wang, C. Y. (2013). The meaning, the sense and the significance: translating the science of mesenchymal stem cells into medicine. Nat. Med. 19, 35-42.

Bouaboula, M., Rinaldi, M., Carayon, P., Carillon, C., Delpech, B., Shire, D., Le Fur, G., and Casellas, P. (1993). Cannabinoid-receptor expression in human leukocytes. Eur. J. Biochem. 214, 173-180.

Broxmeyer, H. E., Orschell, C. M., Clapp, D. W., Hangoc, G., Cooper, S., Plett, P. A., Liles, W. C., Li, X., Graham-Evans, B., Campbell, T. B., et al. (2005). Rapid mobilization of murine and human hematopoietic stem and progenitor cells with AMD3100, a CXCR4 antagonist. J. Exp. Med. 201, 1307-1318.

Chow, A., Lucas, D., Hidalgo, A., Mendez-Ferrer, S., Hashimoto, D., Scheiermann, C., Battista, M., Leboeuf, M., Prophete, C., van Rooijen, N., et al. (2011). Bone marrow CD169+ macrophages promote the retention of hematopoietic stem and progenitor cells in the mesenchymal stem cell niche. J. Exp. Med. 208, 261-271.

Christopher, M. J., Rao, M., Liu, F., Woloszynek, J. R., and Link, D.C. (2011). Expression of the G-CSF receptor in monocytic cells is sufficient to mediate hematopoietic progenitor mobilization by G-CSF in mice. J. Exp. Med. 208, 251-260.

Costola-de-Souza, C., Ribeiro, A., Ferraz-de-Paula, V., Calefi, A. S., Aloia, T. P., Gimenes-Junior, J. A., de Almeida, V. I., Pinheiro, M. L., and Palermo-Neto, J. (2013). Monoacylglycerol lipase (MAGL) inhibition attenuates acute lung injury in mice. PLoS One 8, e77706.

Duerr, G. D., Heinemann, J. C., Suchan, G., Kolobara, E., Wenzel, D., Geisen, C., Matthey, M., Passe-Tietjen, K., Mahmud, W., Ghanem, A., et al. (2014). The endocannabinoid-CB2 receptor axis protects the ischemic heart at the early stage of cardiomyopathy. Basic Res. Cardiol. 109, 425-014-0425-x. Epub 2014 Jul. 1.

Elefteriou, F. (2008). Regulation of bone remodeling by the central and peripheral nervous system. Arch. Biochem. Biophys. 473, 231-236.

Elefteriou, F., Campbell, P., and Ma, Y. (2014). Control of bone remodeling by the peripheral sympathetic nervous system. Calcif. Tissue Int. 94, 140-151.

Fazeli, P. K., Horowitz, M. C., MacDougald, O. A., Scheller, E. L., Rodeheffer, M. S., Rosen, C. J., and Klibanski, A. (2013). Marrow fat and bone—new perspectives. J. Clin. Endocrinol. Metab. 98, 935-945.

Fricker, S. P. (2013). Physiology and pharmacology of plerixafor. Transfus. Med. Hemother 40, 237-245.

Galiegue, S., Mary, S., Marchand, J., Dussossoy, D., Carriere, D., Carayon, P., Bouaboula, M., Shire, D., Le Fur, G., and Casellas, P. (1995). Expression of central and peripheral cannabinoid receptors in human immune tissues and leukocyte subpopulations. Eur. J. Biochem. 232, 54-61.

Galve-Roperh, I., Chiurchiu, V., Diaz-Alonso, J., Bari, M., Guzman, M., and Maccarrone, M. (2013). Cannabinoid receptor signaling in progenitor/stem cell proliferation and differentiation. Prog. Lipid Res. 52, 633-650.

Godlewski, G., Offertaler, L., Wagner, J. A., and Kunos, G. (2009). Receptors for acylethanolamides-GPR55 and GPR119. Prostaglandins Other Lipid Mediat. 89, 105-111.

Guo, L., Amamath, V., and Davies, S. S. (2010). A liquid chromatography-tandem mass spectrometry method for measurement of N-modified phosphatidylethanolamines. Anal. Biochem. 405, 236-245.

Heidt, T., Sager, H. B., Courties, G., Dutta, P., Iwamoto, Y., Zaltsman, A., von Zur Muhlen, C., Bode, C., Fricchione, G. L., Denninger, J., et al. (2014). Chronic variable stress activates hematopoietic stem cells. Nat. Med. 20, 754-758.

Hong, H. S., Lee, J., Lee, E., Kwon, Y. S., Lee, E., Ahn, W., Jiang, M. H., Kim, J. C., and Son, Y. (2009). A new role of substance P as an injury-inducible messenger for mobilization of CD29(+) stromal-like cells. Nat. Med. 15, 425-435.

Hu, C., Yong, X., Li, C., Lu, M., Liu, D., Chen, L., Hu, J., Teng, M., Zhang, D., Fan, Y., and Liang, G. (2013). CXCL12/CXCR4 axis promotes mesenchymal stem cell mobilization to burn wounds and contributes to wound repair. J. Surg. Res. 183, 427-434.

Hwang, J., Adamson, C., Butler, D., Janero, D. R., Makriyannis, A., and Bahr, B. A. (2010). Enhancement of endocannabinoid signaling by fatty acid amide hydrolase inhibition: a neuroprotective therapeutic modality. Life Sci. 86, 615-623.

Iso, Y., Yamaya, S., Sato, T., Poole, C. N., Isoyama, K., Mimura, M., Koba, S., Kobayashi, Y., Takeyama, Y., Spees, J. L., and Suzuki, H. (2012). Distinct mobilization of circulating CD271+ mesenchymal progenitors from hematopoietic progenitors during aging and after myocardial infarction. Stem Cells Trans!. Med. 1, 462-468.

Katayama, Y., Battista, M., Kao, W. M., Hidalgo, A., Peired, A. J., Thomas, S. A., and Frenette, P. S. (2006). Signals from the sympathetic nervous system regulate hematopoietic stem cell egress from bone marrow. Cell 124, 407-421.

Kim, H. Y., and Spector, A. A. (2013). Synaptamide, endocannabinoid-like derivative of docosahexaenoic acid with cannabinoid-independent function. Prostaglandins Leukot. Essent. Fatty Acids 88, 121-125.

Kingsley, P. J., and Marnett, L. J. (2009). Analysis of endocannabinoids, their congeners and COX-2 metabolites. J. Chromatogr. B. Analyt Technol. Biomed. Life. Sci. 877, 2746-2754.

Lan, Y., Kodati, S., Lee, H. S., Omoto, M., Jin, Y., and Chauhan, S. K. (2012). Kinetics and function of mesenchymal stem cells in corneal injury. Invest. Ophthalmol. Vis. Sci. 53, 3638-3644.

Levesque, J. P., Hendy, J., Takamatsu, Y., Simmons, P. J., and Bendall, L. J. (2003). Disruption of the CXCR4/CXCL12 chemotactic interaction during hematopoietic stem cell mobilization induced by GCSF or cyclophosphamide. J. Clin. Invest. 111, 187-196.

Levesque, J. P., and Winkler, I. G. (2008). Mobilization of hematopoietic stem cells: state of the art. Curr. Opin. Organ. Transplant. 13, 53-58.

Li, H., Fong, C., Chen, Y., Cai, G., and Yang, M. (2010). Beta-adrenergic signals regulate adipogenesis of mouse mesenchymal stem cells via cAMP/PKA pathway. Mol. Cell. Endocrinol. 323, 201-207.

Liles, W. C., Rodger, E., Broxmeyer, H. E., Dehner, C., Badel, K., Calandra, G., Christensen, J., Wood, B., Price, T. H., and Dale, D. C. (2005). Augmented mobilization and collection of CD34+ hematopoietic cells from normal human volunteers stimulated with granulocyte?colony-stimulating factor by single-dose administration of AMD3100, a CXCR4 antagonist. Transfusion 45, 295-300.

Martin, C., Bridger, G. J., and Rankin, S. M. (2006). Structural analogues of AMD3100 mobilise haematopoietic progenitor cells from bone marrow in vivo according to their ability to inhibit CXCL12 binding to CXCR4 in vitro. Br. J. Haematol. 134, 326-329.

Mendelson, A., and Frenette, P. S. (2014). Hematopoietic stem cell niche maintenance during homeostasis and regeneration. Nat. Med. 20, 833-846.

Mendez-Ferrer, S., Battista, M., and Frenette, P. S. (2010). Cooperation of beta(2)- and beta(3)-adrenergic receptors in hematopoietic progenitor cell mobilization. Ann. N. Y. Acad. Sci. 1192, 139-144.

Mendez-Ferrer, S., Lucas, D., Battista, M., and Frenette, P. S. (2008). Haematopoietic stem cell release is regulated by circadian oscillations. Nature 452, 442-447.

Mendez-Ferrer, S., Michurina, T. V., Ferraro, F., Mazloom, A. R., Macarthur, B. D., Lira, S. A., Scadden, D. T., Ma'ayan, A., Enikolopov, G. N., and Frenette, P. S. (2010). Mesenchymal and haematopoietic stem cells form a unique bone marrow niche. Nature 466, 829-834.

Morrison, S. J., and Scadden, D. T. (2014). The bone marrow niche for haematopoietic stem cells. Nature 505, 327-334.

Patinkin, D., Milman, G., Breuer, A., Fride, E., and Mechoulam, R. (2008). Endocannabinoids as positive or negative factors in hematopoietic cell migration and differentiation. Eur. J. Pharmacol. 595, 1-6.

Petit, I., Szyper-Kravitz, M., Nagler, A., Lahav, M., Peled, A., Habler, L., Ponomaryov, T., Taichman, R. S., Arenzana-Seisdedos, F., Fujii, N., et al. (2002). G-CSF induces stem cell mobilization by decreasing bone marrow SDF-1 and up-regulating CXCR4. Nat. Immunol. 3, 687-694.

Pitchford, S. C., Furze, R. C., Jones, C. P., Wengner, A. M., and Rankin, S. M. (2009). Differential mobilization of subsets of progenitor cells from the bone marrow. Cell. Stem Cell. 4, 62-72.

Pitchford, S. C., Hahnel, M. J., Jones, C. P., and Rankin, S. M. (2010). Troubleshooting: Quantification of mobilization of progenitor cell subsets from bone marrow in vivo. J. Pharmacol. Toxicol. Methods 61, 113-121.

Riebe, C. J., and Wotjak, C. T. (2011). Endocannabinoids and stress. Stress 14, 384-397.

Rochefort, G. Y., Delorme, B., Lopez, A., Herault, O., Bonnet, P., Charbord, P., Eder, V., and Domenech, J. (2006). Multipotential mesenchymal stem cells are mobilized into peripheral blood by hypoxia. Stem Cells 24, 2202-2208.

Rosen, C. J., Ackert-Bicknell, C., Rodriguez, J. P., and Pino, A. M. (2009). Marrow fat and the bone microenvironment: developmental, functional, and pathological implications. Crit. Rev. Eukaryot. Gene Expr. 19, 109-124.

Scutt, A., and Williamson, E. M. (2007). Cannabinoids stimulate fibroblastic colony formation by bone marrow cells indirectly via CB2 receptors. Calcif. Tissue Int. 80, 50-59.

Sugiura, T. (2009). Physiological roles of 2-arachidonoylglycerol, an endogenous cannabinoid receptor ligand. Biofactors 35, 88-97.

Takahata, Y., Takarada, T., Iemata, M., Yamamoto, T., Nakamura, Y., Kodama, A., and Yoneda, Y. (2009). Functional expression of beta2 adrenergic receptors responsible for protection against oxidative stress through promotion of glutathione synthesis after Nrf2 upregulation in undifferentiated mesenchymal C3H10T1/2 stem cells. J. Cell. Physiol. 218, 268-275.

Tamai, K., Yamazaki, T., Chino, T., Ishii, M., Otsuru, S., Kikuchi, Y., Iinuma, S., Saga, K., Nimura, K., Shimbo, T., et al. (2011). PDGFRalpha-positive cells in bone marrow are mobilized by high mobility group box 1 (HMGB1) to regenerate injured epithelia. Proc. Natl. Acad. Sci. U.S.A. 108, 6609-6614.

Tran, M. A., Dang, T. L., and Berlan, M. (1981). Effects of catecholamines on free fatty acid release from bone marrow adipose tissue. J. Lipid Res. 22, 1271-1276.

Ueda, N., Tsuboi, K., and Uyama, T. (2013). Metabolism of endocannabinoids and related N-acylethanolamines: canonical and alternative pathways. FEBS J. 280, 1874-1894.

Vagima, Y., Lapid, K., Kollet, O., Goichberg, P., Alon, R., and Lapidot, T. (2011). Pathways implicated in stem cell migration: the SDF-1/CXCR4 axis. Methods Mol. Biol. 750, 277-289.

Wang, Y., Johnsen, H. E., Mortensen, S., Bindslev, L., Ripa, R. S., Haack-Sorensen, M., Jorgensen, E., Fang, W., and Kastrup, J. (2006). Changes in circulating mesenchymal stem cells, stem cell homing factor, and vascular growth factors in patients with acute ST elevation myocardial infarction treated with primary percutaneous coronary intervention. Heart 92, 768-774.

1. A method for mobilising haematopoietic stem and progenitor cells (HSPC) and/or mesenchymal stem cells (MSC) in a subject, the method comprising administering a selective beta-3 adrenergic receptor (AR) agonist and an inhibitor of the CXCR4/CXCL12 chemokine axis to the subject.

2. A method for mobilising HSPCs and/or MSCs in a subject, the method comprising administering an inhibitor of the CXCR4/CXCL12 chemokine axis to the subject, wherein the subject is one who is administered a selective beta-3 AR agonist.

3. A method for mobilising HSPCs and/or MSCs in a subject, the method comprising administering a selective beta-3 AR agonist to the subject, wherein the subject is one who is administered an inhibitor of the CXCR4/CXCL12 chemokine axis.

4. A selective beta-3 AR agonist and an inhibitor of the CXCR4/CXCL12 chemokine axis, for use in mobilising HSPCs and/or MSCs in a subject.

5. Use of a selective beta-3 AR agonist and an inhibitor of the CXCR4/CXCL12 chemokine axis, in the manufacture of a medicament for mobilising HSPCs and/or MSCs in a subject.

6. A method for mobilising mesenchymal stem cells (MSC) in a subject, the method comprising administering a beta adrenergic receptor (AR) agonist and an inhibitor of the CXCR4/CXCL12 chemokine axis to the subject.

7. A method for mobilising mesenchymal stem cells (MSC) in a subject, the method comprising administering a beta AR agonist to the subject, wherein the subject is one who is administered an inhibitor of the CXCR4/CXCL12 chemokine axis.

8. A method for mobilising mesenchymal stem cells (MSC) in a subject, the method comprising administering an inhibitor of the CXCR4/CXCL12 chemokine axis to the subject, wherein the subject is one who is administered a beta AR agonist.

9. A method for mobilising haematopoietic stem and progenitor cells (HSPC) and/or mesenchymal stem cells (MSC) in a subject, the method comprising administering a beta adrenergic receptor (AR) agonist and an inhibitor of the CXCR4/CXCL12 chemokine axis to the subject, wherein a beta AR agonist is administered before the inhibitor of the CXCR4/CXCL12 chemokine axis.

10. A method for priming HSPCs and/or MSCs in a subject for mobilisation, the method comprising administering a beta AR agonist to the subject, wherein when the method is for priming HSPCs the beta AR agonist is a selective beta-3 AR agonist.

11. A method or use according to any of the preceding claims, wherein the HSPCs and/or MSCs are mobilised from the bone marrow of the subject.

12. A method or use according to any of the preceding claims, wherein the beta AR agonist is administered chronically to the subject.

13. A method or use according to claim 12, wherein the beta AR agonist is administered at regular intervals over a period of at least one day.

14. A method or use according to any of the preceding claims, wherein the inhibitor of the CXCR4/CXCL12 chemokine axis is administered acutely to the subject.

15. A method or use according to any of the preceding claims, wherein the beta AR agonist is administered at regular intervals over a period of at least one day before the inhibitor of the CXCR4/CXCL12 chemokine axis is administered acutely to the subject, optionally wherein the beta AR agonist is a beta-3 AR agonist.

16. A method or use according to any of the preceding claims, wherein the method or use is for harvesting HSPCs and/or MSCs.

17. A method of obtaining a population of HSPCs and/or MSCs from a subject, the method comprising administering a beta adrenergic receptor (AR) agonist and an inhibitor of the CXCR4/CXCL12 chemokine axis to the subject so as to mobilise the HSPCs and/or MSCs from the bone marrow to the peripheral blood of the subject, and harvesting the mobilised HSPCs and/or MSCs from the peripheral blood;
wherein when the method is for obtaining a population of HSPCs, the beta AR agonist is a selective beta-3 AR agonist.

18. A method of obtaining a population of MSCs from a subject, the method comprising administering a beta adrenergic receptor (AR) agonist to the subject, and harvesting the MSCs from the bone marrow.

19. A method or use according to any of claims 16-18, wherein the harvested HSPCs and/or MSCs are cultivated in vitro.

20. A method or use according to claim 19, wherein the cultivated cells are administered back to the subject or to another subject.

21. A method or use according to any of claims 17-19, wherein the cultivated cells are genetically modified.

22. A method or use according to any of claims 16-21, wherein the harvested cells are stored in a cell bank.

23. A method or use according to any of claims 19-21, wherein the cultivated cells are targeted to damaged tissue in the recipient subject, optionally wherein the damaged tissue is damaged by any one or more of ischaemia, stroke, myocardial infarction, radiotherapy, chemotherapy, auto-immune disease or physical injury.

24. A method for repairing damaged blood vessels, for tissue regeneration, for treating myocardial infarction, stroke, heart disease, peripheral ischaemia, for treating diabetes, autoimmune disease (eg liver cirrhosis, rheumatoid arthritis, sclerederma, graft versus host disease, rejection of organ allografts), systemic lupus erythematosus, multiple sclerosis, cystic fibrosis or other respiratory disease, for immunosuppression, for treating physical injuries (eg sports injuries) or for the healing of chronic wounds, the method comprising the step of administering a population of HSPCs and/or MSCs that has been mobilised and harvested from a subject according to the method of any of claims 17-19, and optionally administering a further therapeutic agent, to a subject.

25. A population of HSPCs and/or MSCs that has been mobilised and harvested from a subject according to the method of any of claims 17-19, and optionally a further therapeutic agent, for use in repairing damaged blood vessels, tissue regeneration, treating myocardial infarction, treating stroke, treating heart disease, treating peripheral ischaemia, treating diabetes, treating autoimmune disease (eg liver cirrhosis, rheumatoid arthritis, sclerederma, graft versus host disease, rejection of organ allografts), treating systemic lupus erythematosus, treating multiple sclerosis, treating cystic fibrosis or other respiratory disease, treating immunosuppression, treating physical injuries (eg sports injuries) or in the healing of chronic wounds; optionally wherein the beta AR agonist is a selective beta AR agonist.

26. Use of a population of HSPCs and/or MSCs that has been mobilised and harvested from a subject according to the method of any of claims 17-19, and optionally a further therapeutic agent, in the manufacture of a medicament for repairing damaged blood vessels, tissue regeneration, treating myocardial infarction, treating stroke, treating heart disease, treating peripheral ischaemia, treating diabetes, treating autoimmune disease (eg liver cirrhosis, rheumatoid arthritis, sclerederma, graft versus host disease, rejection of organ allografts), treating systemic lupus erythematosus, treating multiple sclerosis, treating cystic fibrosis or other respiratory disease, treating immunosuppression, treating physical injuries (eg sports injuries) or for the healing of chronic wounds.

27. A beta AR agonist and an inhibitor of the CXCR4/CXCL12 chemokine axis, and optionally a further therapeutic agent, for use in repairing damaged blood vessels, tissue regeneration, treating myocardial infarction, treating stroke, treating heart disease, treating peripheral ischaemia, treating diabetes, treating autoimmune disease (eg liver cirrhosis, rheumatoid arthritis, sclerederma, graft versus host disease, rejection of organ allografts), treating systemic lupus erythematosus, treating multiple sclerosis, treating cystic fibrosis or other respiratory disease, treating immunosuppression, treating physical injuries (eg sports injuries) or for the healing of chronic wounds, optionally wherein the beta AR agonist is a selective beta-3 AR agonist.

28. Use of a beta AR agonist and an inhibitor of the CXCR4/CXCL12 chemokine axis, and optionally a further therapeutic agent, in the manufacture of a medicament for repairing damaged blood vessels, tissue regeneration, treating myocardial infarction, treating stroke, treating heart disease, treating peripheral ischaemia, treating diabetes, treating autoimmune disease (eg liver cirrhosis, rheumatoid arthritis, sclerederma, graft versus host disease, rejection of organ allografts), treating systemic lupus erythematosus, treating multiple sclerosis, treating cystic fibrosis or other respiratory disease, treating immunosuppression, treating physical injuries (eg sports injuries) or for the healing of chronic wounds, optionally wherein the beta AR agonist is a selective beta-3 AR agonist.

29. A method for repairing damaged blood vessels, for tissue regeneration, for treating myocardial infarction, stroke, heart disease, peripheral ischaemia, for treating diabetes, autoimmune disease (eg liver cirrhosis, rheumatoid arthritis, sclerederma, graft versus host disease, rejection of organ allografts), systemic lupus erythematosus, multiple sclerosis, cystic fibrosis or other respiratory disease, for immunosuppression, for treating physical injuries (eg sports injuries) or for the healing of chronic wounds, the method comprising the step of administering a beta AR agonist and an inhibitor of the CXCR4/CXCL12 chemokine axis, and optionally a further therapeutic agent, to a subject, optionally wherein the beta AR agonist is a selective beta-3 AR agonist.

30. A use according to claim 27 or 28, or a method according to claim 29, wherein administration of the beta AR agonist and an inhibitor of the CXCR4/CXCL12 chemokine axis mobilises HSPCs and/or MSCs in the subject.

31. A population of HSPCs and/or MSCs isolated from a subject, wherein the subject has been administered a beta AR agonist and optionally an inhibitor of the CXCR4/CXCL12 chemokine axis, optionally wherein the beta AR agonist is a selective beta-3 AR agonist.

32. A kit of parts comprising (i) a selective beta-3 AR agonist and (ii) an inhibitor of the CXCR4/CXCL12 chemokine axis.

33. A method for stimulating proliferation of MSCs, the method comprising contacting the MSCs with a beta AR agonist.

34. A method according to claim 33, wherein the beta AR agonist is a selective beta-3 AR agonist.

35. A method according to claim 34 or 35, wherein the method is performed in vivo or in vitro.

36. A method according to claim 35, wherein the method is performed in vitro and the cells are cultivated in vitro.

37. A method according to any of claims 1-3, 6-24, 29-30, and 33-36, a use according to any of claims 4, 5, 11-16, 19-23, and 25-28 and 30, a population of HSPCs and/or MSCs according to claim 31, and a kit of parts according to claim 32, wherein the beta AR agonist is a selective beta-3 AR agonist such as any of BRL37344; Mirabegron; CL316243; L-742,791; L-796,568; LY-368,842; Mirabegron (YM-178); Ro40-2148; Solabegron (GW-427,353); Betanis (Astellas); Betmiga (Astellas); Myrbetriq (Astellas); TT-138 (Mitsubishi Tanabe Pharma); GS-332 (Mitsubishi Tanabe Pharma); MN-246 (Mitsubishi Tanabe Pharma); FMP-1970302 (Molecular Design); and 4SC (4sc discovery).

38. A method according to any of claims 6-24, 29-30, and 33-36, a use according to any of claims 4, 5, 11-16, 19-23, 25-28 and 30, a population of HSPCs and/or MSCs according to claim 31, and a kit of parts according to claim 32, wherein the beta AR agonist is a general beta AR agonist such as any of isoproterenol, epinephrine and norepinephrine.

39. A method according to any of claims 1-3, 6-24, 29-30, and 33-38, a use according to any of claims 4, 5, 11-16, 19-23, 25-28, 30, 37 and 38, a population of HSPCs and/or MSCs according to any of claims 31, 37 and 38, and a kit of parts according to any of claims 32, 37 and 38, wherein the inhibitor of the CXCR4/CXCL12 chemokine axis is a an agent that reduces the synthesis or function of CXCL12.

40. A method according to any of claims 1-3, 6-24, 29-30, and 33-38, a use according to any of claims 4, 5, 11-16, 19-23, 25-28, 30, and 37-39, a population of HSPCs and/or MSCs according to any of claims 31, and 37-39, and a kit of parts according to any of claims 32 and 37-39, wherein the inhibitor of the CXCR4/CXCL12 chemokine axis is a an antagonist of CXCR4.

41. A method, use, population, kit of parts or composition according to claim 40, wherein the antagonist of CXCR4 is AMD3100 or KRH3955.

42. A method or use for mobilising HSPCs and/or MSCs, according to any of claims 1-29 and 33-40 wherein the method or use further comprises administering a COX inhibitor (eg Naproxen, Aspirin) and/or a Free Fatty Acid Hydrolyse (FFAH) inhibitor (eg URB597) to the subject.

43. A method of identifying an inhibitor of the CXCR4/CXCL12 chemokine axis in a subject, wherein the method comprises the steps of: (i) administering a beta AR agonist to the subject; (ii) administering a test agent to the subject; and (iii) assessing the amount of HSPCs and/or MSCs in a sample from the subject.

44. A method of identifying an agonist of the beta AR receptor in a subject, wherein the method comprises the steps of: (i) administering a test agent to the subject (ii) administering an inhibitor of the CXCR4/CXCL12 chemokine axis to the subject; and (iii) assessing the amount of HSPCs and/or MSCs in a sample from the subject.
